(19) United States
(12) Patent Application Publication
(10) Pub. No.: US 2026/0022170 A1
(43) Pub. Date: Jan. 22, 2026

(54) ANTI-SIGLEC-9 ANTIBODIES AND USES THEREOF

(71) Applicants: ADAPTAM THERAPEUTICS S.L, San Sebastian (ES); ASOCIACIÓN CENTRO DE INVESTIGACIÓN COOPERATIVA EN BIOCIENCIAS-CIC bioGUNE, Derio (ES)

(72) Inventors: Francisco de Asís Palazón García, San Sebastián (ES); Alexandre Bosch Martínez, Berango (ES)

(21) Appl. No.: 19/201,688

(22) Filed: May 7, 2025

(30) Foreign Application Priority Data
May 10, 2024 (EP) 24382509.8

(51) Int. Cl. C07K 16/28; A61K 39/395; A61K 47/68; A61P 35/00

(52) U.S. Cl. CPC C07K 16/2803; A61K 39/39558; A61K 47/6803; A61K 47/6849; A61P 35/00; C07K 2317/24; C07K 2317/34; C07K 2317/52; C07K 2317/73; C07K 2317/76; C07K 2317/92; C07K 2317/94; C07K 2319/21

(57) ABSTRACT

The present invention provides anti-Siglec-9 antibodies and antigen-binding fragments thereof as well as isolated nucleic acids, vectors, engineered cells, formulations thereof, and methods of use thereof for treating diseases including cancer and bone disease in a human subject. The invention is further directed to bispecific and multispecific antibodies, antibody-drug conjugates and chimeric antigen receptors derived from the anti-Siglec-9 antibodies and fragments thereof.

### CROSS-REFERENCE TO RELATED APPLICATIONS

This application claims the benefit of priority of European Patent Application No. EP24382509.8, filed May 10, 2024, which is incorporated herein by reference in its entirety.

### SEQUENCE LISTING

This application contains a Sequence Listing which has been filed electronically in compliance with ST.26 format and is hereby incorporated by reference in its entirety. The Sequence Listing, created on May 5, 2025, is named 5337_002PC01_SequenceListing_ST26.xml, and is 24,391 bytes in size.

### BACKGROUND

Sialic acid-binding Ig-like lectin-9 (Siglec-9), is a type 1, immunoglobulin-like, transmembrane protein expressed on immune and hematopoietic cells associated with multiple human diseases including, autoimmunity, susceptibility to infection, multiple types of cancer including lymphoma, leukemia and acute myeloid leukemia, systemic lupus erythematosus, rheumatoid arthritis, neurodegenerative disorders, asthma, allergy, sepsis, chronic obstructive pulmonary disease, graft-versus-host disease, eosinophilia, and osteoporosis. In solid cancers, epithelial tumor cells produce heavily glycosylated mucins that bind Siglec-9, suggesting that blocking the increased ligand interactions would be therapeutically beneficial (Ohta et al. (2010) Biochem. and Biophys. Res. Comm. 402: 663-669; Belisle et al. (2010) Mol. Cancer 9:118).

Accordingly, there is a need for therapeutic antibodies that specifically bind to Siglec-9 and reduce or block interactions between Siglec-9 and one or more Siglec-9 ligands, and/or modulate one or more Siglec-9-dependent biological functions in order to treat one or more diseases, disorders, and conditions including cancer.

### SUMMARY

Provided herein is an antibody or antigen-binding fragment thereof that binds to Siglec-9 protein, wherein the antibody or antigen-binding fragment thereof comprises a heavy chain variable complementarity determining region 1 (CDR-H1) comprising the amino acid sequence of SEQ ID NO: 5, a CDR-H2 comprising the amino acid sequence of SEQ ID NO: 6, a CDR-H3 comprising the amino acid sequence of SEQ ID NO: 7, a light chain variable complementarity determining region 1 (CDR-L1) comprising the amino acid sequence of SEQ ID NO: 8, a CDR-L2 comprising the amino acid sequence of SEQ ID NO: 9, and a CDR-L3 comprising the amino acid sequence of SEQ ID NO: 10; or a heavy chain variable complementarity determining region 1 (CDR-H1) comprising the amino acid sequence of SEQ ID NO: 13, a CDR-H2 comprising the amino acid sequence of SEQ ID NO: 14, a CDR-H3 comprising the amino acid sequence of SEQ ID NO: 15, a light chain variable complementarity determining region 1 (CDR-L1) comprising the amino acid sequence of SEQ ID NO: 16, a CDR-L2 comprising the amino acid sequence of SEQ ID NO: 17, and a CDR-L3 comprising the amino acid sequence of SEQ ID NO:18.

In one embodiment, the antibody or antigen-binding fragment thereof comprises a variable heavy chain (VH) comprising the amino acid sequence of SEQ ID NO: 3; a variable heavy chain (VH) comprising the amino acid sequence of SEQ ID NO: 11; or a variable heavy chain (VH) comprising the amino acid sequence of SEQ ID NO: 19.

In one embodiment, the antibody or antigen-binding fragment thereof comprises a variable light chain (VL) comprising the amino acid sequence of SEQ ID NO: 4; or a variable light chain (VL) comprising the amino acid sequence of SEQ ID NO: 12; or a variable light chain (VL) comprising the amino acid sequence of SEQ ID NO: 20.

In one embodiment, the antibody or antigen-binding fragment thereof comprises a heavy chain variable region and a light chain variable region comprising the amino acid sequences of SEQ ID NOs: 3 and 4, respectively; a heavy chain variable region and a light chain variable region comprising the amino acid sequences of SEQ ID NOs: 11 and 12, respectively; or a heavy chain variable region and a light chain variable region comprising the amino acid sequences of SEQ ID NOs: 19 and 20, respectively.

In one embodiment, the antibody or antigen-binding fragment thereof is of the IgG class, the IgM class, or the IgA class.

In one embodiment, the antibody or antigen-binding fragment thereof has an IgGI, IgG2, IgG3, IgG4, IgA, or IgA2 isotype.

In one embodiment, the antibody or antigen-binding fragment thereof comprises the CDR-H1, CDR-H2, CDR-H3, CDR-L1, CDR-L2, and CDR-L3 of an antibody selected from the group consisting of ED2D5 and CB6B3.

Also provided herein in an antibody or antigen-binding fragment thereof that binds to the same epitope of Siglec-9 protein as the antibody or antigen-binding fragment thereof provided herein.

In one embodiment, the antibody or antigen-binding fragment thereof is a murine antibody, a humanized antibody, a bispecific antibody, a monoclonal antibody, a multivalent antibody, a conjugated antibody, a human antibody, or a chimeric antibody or antigen-binding fragment thereof.

In one embodiment, the antibody or antigen-binding fragment blocks the binding of Siglec-9 protein to sialic acid.

Also provided herein is a polynucleotide comprising a nucleic acid molecule encoding the VH of the antibody or antigen-binding fragment thereof provided herein.

Also provided herein is a polynucleotide comprising a nucleic acid molecule encoding the VL of the antibody or antigen-binding fragment thereof provided herein.

Also provided herein is a polynucleotide comprising a nucleic acid molecule encoding the VH and the VL of the antibody or antigen-binding fragment thereof provided herein.

Also provided herein is an isolated vector comprising one or more of the polynucleotides provided herein.

Also provided herein is a host cell comprising one or more of the polynucleotides provided herein, the vector provided herein, or a first vector comprising a polynucleotide provided herein and a second vector comprising a polynucleotide provided herein.

Also provided herein is a method of producing an antibody or antigen-binding fragment thereof comprising culturing the host cell provided herein under conditions such that the antibody or antigen-binding fragment thereof is produced.

Also provided herein is a method of blocking the binding of Siglec-9 protein to sialic acid in cells comprising contacting the cells with the antibody or antigen-binding fragment thereof provided herein.

In one embodiment, the cells are selected from the group consisting of dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia.

Also provided herein is a method of inhibiting one or more activities of Siglec-9 protein in cells comprising contacting the cells with the antibody or antigen-binding fragment thereof provided herein.

In one embodiment, the cells are selected from the group consisting of dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia.

Also provided herein is a method for detecting Siglec-9 protein in a sample comprising contacting the sample with the antibody or antigen-binding fragment thereof provided herein

Also provided herein is a pharmaceutical composition comprising the antibody or antigen-binding fragment thereof of provided herein and a pharmaceutically acceptable carrier.

Also provided herein is a method of treating or preventing a Siglec-9-associated disorder in a subject in need thereof comprising administering to the subject a therapeutically effect amount of the antibody or antigen-binding fragment thereof provided herein.

In one embodiment, the disorder is selected from the group consisting of infection, inflammation, sepsis, rheumatoid arthritis, pulmonary disease, neurodegeneration, and autoimmune disease.

In one embodiment, the disorder is characterized by cells with increased levels of sialic acid ligands of Siglec-9 protein on their surface compared to a normal cell without the disorder.

Also provided herein is a method of treating or preventing cancer in a subject in need thereof comprising administering to the subject a therapeutically effect amount of the antibody or antigen-binding fragment thereof provided herein.

In one embodiment, the cancer is selected from the group consisting of bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), and multiple myeloma.

In one embodiment, the cancer is characterized by malignant cells with increased levels of sialic acid ligands of Siglec-9 protein on their surface compared to a non-malignant cell.

In one embodiment, the antibody or antigen-binding fragment is administered in combination with an additional therapeutic agent for treating, preventing and/or diagnosing the disorder or cancer.

In one embodiment, the therapeutic agent is a chemotherapeutic agent or an immunotherapy.

In one embodiment, the chemotherapeutic agent is selected from the group consisting of alkylating agents, antimetabolites, antitumor antibiotics, topoisomerase inhibitors, mitotic inhibitors, platinum-based drugs, and any combination thereof.

In one embodiment, the immunotherapy targets one or more target proteins selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIMI, TIM3, TIM4, A2AR, LAG3, DR-5, CD2, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof.

In one embodiment, the antibody or antigen-binding fragment and therapeutic agent are administered sequentially.

In one embodiment, the antibody or antigen-binding fragment and therapeutic agent are administered simultaneously.

Also provided herein is a kit comprising the antibody or antigen-binding fragment thereof provided herein, a detection reagent, and instructions for use for detection of a Siglec-9 antigen.

In one embodiment, the Siglec-9 protein is human Siglec-9 protein.

Also provided herein is a bispecific antibody comprising a first antigen-binding portion that binds to Siglec-9 comprising a heavy chain variable complementarity determining region 1 (CDR-H1) comprising the amino acid sequence of SEQ ID NO: 5, a CDR-H2 comprising the amino acid sequence of SEQ ID NO: 6, a CDR-H3 comprising the amino acid sequence of SEQ ID NO: 7, a light chain variable complementarity determining region 1 (CDR-L1) comprising the amino acid sequence of SEQ ID NO: 8, a CDR-L2 comprising the amino acid sequence of SEQ ID NO: 9, and a CDR-L3 comprising the amino acid sequence of SEQ ID NO: 10; or a heavy chain variable complementarity determining region 1 (CDR-H1) comprising the amino acid sequence of SEQ ID NO: 13, a CDR-H2 comprising the amino acid sequence of SEQ ID NO: 14, a CDR-H3 comprising the amino acid sequence of SEQ ID NO: 15, a light chain variable complementarity determining region 1 (CDR-L1) comprising the amino acid sequence of SEQ ID NO: 16, a CDR-L2 comprising the amino acid sequence of SEQ ID NO: 17, and a CDR-L3 comprising the amino acid sequence of SEQ ID NO:18; and a second antigen-binding portion that binds to a second antigen.

In one embodiment, the first antigen-binding portion comprises a variable heavy chain (VH) comprising the amino acid sequence of SEQ ID NO: 3; a variable heavy chain (VH) comprising the amino acid sequence of SEQ ID NO: 11; or a variable heavy chain (VH) comprising the amino acid sequence of SEQ ID NO: 19.

In one embodiment, the first antigen-binding portion comprises a variable light chain (VL) comprising the amino acid sequence of SEQ ID NO: 4; or a variable light chain (VL) comprising the amino acid sequence of SEQ ID NO: 12; or a variable light chain (VL) comprising the amino acid sequence of SEQ ID NO: 20.

In one embodiment, the first antigen-binding portion comprises a heavy chain variable region and a light chain variable region comprising the amino acid sequences of SEQ ID NOs: 3 and 4, respectively; a heavy chain variable region and a light chain variable region comprising the amino acid sequences of SEQ ID NOs: 11 and 12, respectively; or heavy chain variable region and a light chain variable region comprising the amino acid sequences of SEQ ID NOs: 19 and 20, respectively.

In one embodiment, the second antigen is a cell surface protein that is enriched on blood-brain barrier endothelial cells selected from the group consisting of transferrin receptor, insulin receptor, insulin-like growth factor receptor, low-density lipoprotein receptor related proteins 1 and 2, diphtheria toxin receptor, CRM197, a llama single domain antibody, TMEM 30(A), a protein transduction domain, TAT, Syn-B, penetratin, a poly-arginine peptide, Angiopep peptides, other cell surface proteins that are enriched on blood-brain barrier endothelial cells.

In one embodiment, the second antigen is a protein expressed on immune cells selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIMI, TIM3, TIM4, A2AR, LAG3, DR-5, CD2, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDOl, STING, and phosphatidylserine.

In one embodiment, the second antigen is also Siglec-9.

In one embodiment, each of the first and second antigen-binding portions thereof is independently from a murine antibody, a humanized antibody, a monoclonal antibody, a conjugated antibody, a human antibody, or a chimeric antibody or antigen-binding fragment thereof.

In one embodiment, the antibody blocks the binding of Siglec-9 protein to sialic acid.

Also provided herein is bispecific T-cell engager comprising one binding domain that specifically binds to Siglec-9 on target cancer cells, and a second binding domain that specifically engages CD3 on T cells.

In one embodiment, the binding domain for Siglec-9 comprises any of the antibody or antigen-binding fragments thereof provided herein, and the CD3 binding domain is designed to activate T cells upon binding.

Also provided herein is a bispecific antibody or antibody fragment construct that simultaneously targets Siglec-9 and Siglec-15, wherein one arm of the bispecific construct is specifically directed against Siglec-9, and the other arm is specifically directed against Siglec-15.

In one embodiment, the binding domain for Siglec-9 comprises any of the antibody or antigen-binding fragments thereof provided herein.

Also provided herein is a bispecific antibody or antibody fragment construct that simultaneously targets Siglec-9 and PD-L1, wherein one arm of the bispecific construct is directed against Siglec-9, and the other arm is directed against PD-L1.

In one embodiment, the binding domain for Siglec-9 comprises any of the antibody or antigen-binding fragments of claims 1-10.

Also provided herein is a polynucleotide comprising a nucleic acid molecule encoding the first antigen-binding portion provided herein.

Also provided herein is a polynucleotide comprising a nucleic acid molecule encoding the second antigen-binding portion provided herein.

Also provided herein is an isolated vector comprising a polynucleotide provided herein.

Also provided herein is an isolated vector comprising the polynucleotide provided herein.

Also provided herein is a host cell comprising a first vector provided herein and a second vector provided herein.

Also provided herein is a method of producing a bispecific antibody comprising culturing the host cell provided herein under conditions such that the bispecific antibody is produced.

Also provided herein is a method of blocking the binding of Siglec-9 protein to sialic acid in cells comprising contacting the cells with the bispecific antibody provided herein.

In one embodiment, the cells are selected from the group consisting of dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia.

Also provided herein is a method of inhibiting one or more activities of Siglec-9 protein in cells comprising contacting the cells with the bispecific antibody provided herein.

In one embodiment, the cells are selected from the group consisting of dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia.

Also provided herein is a method for detecting Siglec-9 protein in a sample comprising contacting the sample with the bispecific antibody provided herein.

Also provided herein is a pharmaceutical composition comprising the bispecific antibody provided herein and a pharmaceutically acceptable carrier.

Also provided herein is a method of treating or preventing a Siglec-9-associated disorder in a subject in need thereof comprising administering to the subject a therapeutically effect amount of the bispecific antibody provided herein.

In one embodiment, the disorder is selected from the group consisting of infection, inflammation, sepsis, rheumatoid arthritis, pulmonary disease, neurodegeneration, and autoimmune disease.

In one embodiment, the disorder is characterized by cells with increased levels of sialic acid ligands of Siglec-9 protein on their surface compared to a normal cell without the disorder.

Also provided herein is a method of treating or preventing cancer in a subject in need thereof comprising administering to the subject a therapeutically effect amount of the bispecific antibody provided herein.

In one embodiment, the cancer is selected from the group consisting of bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CMIL), and multiple myeloma, thyroid cancer, gastric cancer, esophageal cancer, hepatocellular carcinoma, gallbladder cancer, soft tissue sarcoma, head and neck cancer, cervical cancer, testicular cancer, mesothelioma, glioblastoma, neuroendocrine tumors, uterine sarcoma, adrenocortical carcinoma, and stomach adenocarcinoma.

In one embodiment, the cancer is characterized by malignant cells with increased levels of sialic acid ligands of Siglec-9 protein on their surface compared to a non-malignant cell.

In one embodiment, the bispecific antibody is administered in combination with an additional therapeutic agent for treating, preventing and/or diagnosing the disorder or cancer.

In one embodiment, the therapeutic agent is a chemotherapeutic agent or an immunotherapy.

In one embodiment, the immunotherapy targets one or more target proteins selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIMI, TIM3, TIM4, A2AR, LAG3, DR-5, CD2, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof.

In one embodiment, the bispecific antibody and therapeutic agent are administered sequentially.

In one embodiment, the bispecific antibody and therapeutic agent are administered simultaneously.

Also provided herein is a conjugate comprising an antibody or antigen-binding fragment thereof that binds to Siglec-9 protein coupled to an active agent.

In one embodiment, the antibody or antigen-binding fragment thereof comprises the antibody or antigen-binding fragment provided herein.

In one embodiment, conjugate comprises a linker coupling the antibody or antigen-binding fragment to the active agent.

In one embodiment, the linker is selected from the group consisting of an acid labile linker, a disulfide linker, a protected disulfide linker, an ester linker, an ortho ester linker, a phosphonamide linker, a biocleavable peptide linker, an azo linker, a hydrazone linker, a cathepsin-B cleavable linker, a b-d-glucuronide linker, a non-cleavable linker, an SPDB linker, an SMPB linker, a hydrophylic linker, a self-immolative linker, and an aldehyde bond.

In one embodiment, the active agent is an immunomodulatory agent, a cytotoxic agent, a radiolabeled agent, or any combination thereof.

Also provided herein is a method of producing the conjugate provided herein, comprising reacting the antibody or antigen-binding fragment, the linker, and the active agent under conditions such that the conjugate is produced.

Also provided herein is a pharmaceutical composition comprising the conjugate provided herein and a pharmaceutically acceptable carrier.

Also provided herein is a method of treating or preventing a Siglec-9 associated disorder in a subject in need thereof comprising administering to the subject a therapeutically effect amount of the conjugate provided herein.

In one embodiment, the disorder is selected from the group consisting of infection, inflammation, sepsis, rheumatoid arthritis, pulmonary disease, neurodegeneration, and autoimmune disease.

In one embodiment, the disorder is characterized by cells with increased levels of sialic acid ligands of Siglec-9 protein on their surface compared to a normal cell without the disorder.

Also provided herein is a method of treating or preventing cancer in a subject in need thereof comprising administering to the subject a therapeutically effect amount of the conjugate provided herein.

In one embodiment, the cancer is selected from the group consisting of bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), and multiple myeloma.

In one embodiment, the cancer is characterized by malignant cells with increased levels of sialic acid ligands of Siglec-9 protein on their surface compared to a non-malignant cell.

In one embodiment, the conjugate is administered in combination with an additional therapeutic agent for treating, preventing and/or diagnosing the disorder or cancer.

In one embodiment, the therapeutic agent is a chemotherapeutic agent or an immunotherapy.

In one embodiment, the immunotherapy targets one or more target proteins selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIMI, TIM3, TIM4, A2AR, LAG3, DR-5, CD2, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof.

In one embodiment, the conjugate and therapeutic agent are administered sequentially.

In one embodiment, the conjugate and therapeutic agent are administered simultaneously.

Also provided herein is an isolated chimeric antigen receptor (CAR) comprising an extracellular portion comprising an antibody or antigen-binding fragment that binds to Siglec-9 protein comprising the antibody or antigen-binding fragment provided herein; a transmembrane domain; and an intracellular signaling domain.

In one embodiment, the intracellular domain comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

Also provided herein is a polynucleotide comprising a nucleic acid molecule encoding the CAR provided herein.

Also provided herein is a cell expressing the CAR provided herein.

In one embodiment, the cell is an immune cell.

In one embodiment, the intracellular domain comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

Also provided herein is a pharmaceutical composition comprising the CAR provided herein or the cell provided herein and a pharmaceutically acceptable carrier.

Also provided herein is a method of treating or preventing a Siglec-9 associated disorder in a subject in need thereof comprising administering to the subject a therapeutically effect amount of the CAR provided herein or the cell provided herein.

In one embodiment, the disorder is selected from the group consisting of infection, inflammation, sepsis, rheumatoid arthritis, pulmonary disease, neurodegeneration, and autoimmune disease.

In one embodiment, the disorder is characterized by cells with increased levels of sialic acid ligands of Siglec-9 protein on their surface compared to a normal cell without the disorder.

Also provided herein is a method of treating or preventing cancer in a subject in need thereof comprising administering to the subject a therapeutically effect amount of the CAR provided herein or the cell provided herein.

In one embodiment, the cancer is selected from the group consisting of bladder cancer, brain cancer, breast cancer, colon cancer, rectal cancer, endometrial cancer, kidney cancer, renal cell cancer, renal pelvis cancer, leukemia, lung cancer, melanoma, non- Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), and multiple myeloma.

In one embodiment, the cancer is characterized by malignant cells with increased levels of sialic acid ligands of Siglec-9 protein on their surface compared to a non-malignant cell.

Also provided herein is chimeric antigen receptor (CAR) T-cell engineered to secrete anti-Siglec-9 antibodies or antibody fragments, wherein the CAR T-cell targets include, but are not limited to, CD19 for B-cell malignancies, BCMA for multiple myeloma, CD22 for acute lymphoblastic leukemia, CD30 for Hodgkin lymphoma, CD33 for acute myeloid leukemia, HER2 for breast and other HER2+ cancers, GD2 for neuroblastoma, GPC3 for hepatocellular carcinoma, and mesothelin for mesothelioma and ovarian cancer.

Also provided herein is a method for prognostic assessment of a subject diagnosed with cancer, comprising determining the level of Siglec-9 expression in a tumor sample of the subject using an anti-Siglec-9 antibody or fragment thereof provided herein, wherein a higher level of Siglec-9 expression is indicative of a poorer prognosis.

In one embodiment, the method further comprises selecting a treatment regimen based on the determined level of Siglec-9 expression.

Also provided herein is a method for monitoring the efficacy of a treatment in a subject, comprising measuring changes in Siglec-9 expression in biological samples from the subject over time using an anti-Siglec-9 antibody or fragment thereof provided herein, wherein a decrease in Siglec-9 expression is indicative of a positive treatment response.

Also provided herein is a method of increasing IFN-γ production comprising contacting cells with the antibody or antigen-binding fragment thereof provided herein.

Also provided herein is a method of promoting CAR-T cytotoxicity comprising providing the antibody or antigen-binding fragment thereof provided herein.

### BRIEF DESCRIPTION OF THE FIGURES

FIGS. 1A and 1B show the binding of a panel of supernatants from hybridomas obtained from BALB/c mice immunized with recombinant Siglec-9 ectodomain as immunogen.

FIG. 1C shows the binding of a panel of supernatants from hybridomas obtained from C57/bl6 mice immunized with the partial V domain.

FIG. 1D shows a schematic of domains corresponding to human recombinant Siglec-9.

FIG. 2A shows a dot plot of the total peripheral blood mononuclear cells (PBMC).

FIG. 2B shows a dot plot of monocytic cells (CD14+) expressing Siglec-9 on their surface.

FIG. 2C shows histogram of the binding of the selected antibodies compared to a commercial anti-Siglec-9 antibody (clone K8) and negative control (FMO), quantified by staining with an anti-IgGI isotype mouse-BV421 antibody.

FIG. 3 shows dot plots of the binding capacity of selected clones to Siglec-9, quantified by staining with an Fc-specific antibody.

FIG. 4 shows a chart of the percentage of blocking of the binding of recombinant Siglec-9-FC (4 pg/mL) to Ramos lymphoma cells measured by flow cytometry.

FIG. 5 shows graphs of the quantification of the affinity of the anti-Siglec-9 clones.

FIG. 6A shows flow cytometry histograms of the binding of recombinant Siglec-9-Fc to Ramos lymphoma cells.

FIG. 6B shows dose-response curves showing the blocking capacity of the indicated anti-Siglec-9 mAbs.

FIG. 7 shows SDS-PAGE analysis of the different antibodies under reducing and non-reducing conditions.

FIG. 8 shows Size-exclusion chromatography of the different antibodies using HPLC.

FIG. 9A shows binding of mouse IgG CB6B3 to immobilized human Siglec-9 molecule, analyzed via ELISA.

FIG. 9B shows binding of different IgG CB6B3 to immobilized human Siglec-9 molecule, analyzed via ELISA.

FIG. 10A shows representative biolayer interferometry (BLI) sensograms for anti-Siglec-9 antibodies in Fab format at 250 nM binding to Siglec-3, Siglec-6, Siglec-7, Siglec-8, Siglec-9 and Siglec-10.

FIG. 10B shows the binding of mAb clones ED2D5, CB6B3, BC3G5, and DH1A11 to ectodomains of Siglec-9 and Siglec-7 via ELISA.

FIG. 11 shows the binding of mAb huCB6B3, benchark antibodies, and an isotype control to recombinant human Siglec-9 protein via ELISA. EC50 values are shown in the table. Benchmark-1: clone mAbA; Benchmark-2: clone 5C6, Benchmark-3: clone 68D4, Benchmark-4: clone Tf73.

FIG. 12A shows a schematic of CD14+ monocyte differentiation and polarization into M1- or M2-like macrophages.

FIG. 12B shows representative flow cytometry histograms showing the surface expression of Siglec-9, PD-L1, and CD206 on polarized macrophages.

FIG. 12C shows quantification of CD206, PD-Li, and Siglec-9 surface expression on polarized macrophage subsets in mean fluorescence intensity (MFI).

FIG. 13A shows representative flow cytometry histograms showing binding of anti-Siglec-9 mAb clone huCB6B3 and benchmark mAbs to human M1-like macrophages. Benchmark-1: clone mAbA; Benchmark-2: clone 5C6, Benchmark-3: clone 68D4, Benchmark-4: clone Tf73.

FIG. 13B shows representative flow cytometry histograms showing binding of anti-Siglec-9 mAb clone huCB6B3 and benchmark mAbs to human M2-like macrophages. Benchmark-1: clone mAbA; Benchmark-2: clone 5C6, Benchmark-3: clone 68D4, Benchmark-4: clone Tf73.

FIG. 14A shows a schematic of T cell co-culture with THP-1 S9⁺ cells in the presence of an anti-Siglec-9 mAb.

FIG. 14B shows IFN-γ levels measured by ELISA in supernatants following a 72 hour incubation of human T cells co-cultured with THP-1 S9⁺ cells, anti-Siglec-9 mAb huCB6B3.

FIG. 15A shows a schematic of THP-1 target cells expressing CD19 and Siglec-9 co-cultured with anti-CD19 CAR-T cells in the presence of an anti-Siglec-9 antibody. The bar graph shows the cytotoxic activity of 19BBz CAR-T cells against THP1-CD19-S9 cells in the presence of antibody huCB6B3 or a control after 16 hours of co-culture.

FIG. 15B shows a schematic of THP-1 target cells expressing NY-ESO and Siglec-9 co-cultured with anti-NY-ESO-1 TCR-engineered human T cells in the presence of an anti-Siglec-9 antibody. The bar graph shows the cytotoxic activity of anti-NY-ESO-1 specific T cells against THP1-NY-ESO-1-S9 cells in the presence of antibody huCB6B3 or a control after 16 hours of co-culture.

FIG. 16A shows a schematic of co-culture of CAR-T cells, human macrophages, and MDA-MB-231-CD19 tumor cells in the presence of an anti-Siglec-9 mAb.

FIG. 16B shows a bar graph of CAR-T cytotoxicity against MDA-MB-231-CD19 tumor cells in the presence of anti-Siglec-9 mAb huCB6B3 or a human IgG control.

FIG. 17 shows expression of Siglec-9, CD163, and PD-L1 by IHC staining in serial sections of a human hepatocellular carcinoma sample. Bars in the images correspond to 100 μm.

FIG. 18 shows expression of Siglec-9, CD163, and PD-L1 by IHC staining in serial sections of a human breast cancer sample. Bars in the images correspond to 100 μm.

FIG. 19 shows expression of Siglec-9, CD163, and PD-L1 by IHC staining in serial sections of a human lung adenocarcinoma sample. Bars in the images correspond to 100 μm.

FIG. 20 shows expression of Siglec-9, CD163, and PD-L1 by IHC staining in serial sections of a human rectum adenocarcinoma sample. Bars in the images correspond to 100 μm.

### DETAILED DESCRIPTION

The headings provided herein are not limitations of the various embodiments of the disclosure, which can be defined by reference to the specification as a whole. It is also to be understood that the terminology used herein is for the purpose of describing particular embodiments only, and is not intended to be limiting, since the scope of the present disclosure will be limited only by the appended claims.

### A. Terminology

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The indefinite articles “a” and “an” to describe an element or component means that one or at least one of these elements or components is present. Although these articles are conventionally employed to signify that the modified noun is a singular noun, as used herein the articles “a” and “an” also include the plural, unless otherwise stated in specific instances. Similarly, the definite article “the,” as used herein, also signifies that the modified noun can be singular or plural, again unless otherwise stated in specific instances.

As used herein, the term “about” means within an acceptable error range for the particular value as determined by one of ordinary skill in the art, which depends in part on how the value is measured or determined, i.e., the limitations of the measurement system. “About” used with numerical values means “within 10% of the stated value,” unless expressly noted otherwise. For example, “about 5% by weight” means from 4.5% by weight to 5.5% by weight.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” and “comprising,” are open-ended terms meaning “including, but not limited to.” To the extent a given embodiment disclosed herein “comprises” certain elements, it should be understood that the present disclosure also specifically contemplates and discloses embodiments that “consist essentially of” those elements and that “consist of” those elements.

As used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Any numerical range recited herein is intended to include all sub-ranges subsumed therein. Where a range of numerical values is recited herein, comprising upper and lower values, unless otherwise stated in specific circumstances, the range is intended to include the endpoints thereof, and all integers and fractions within the range. It is not intended that the scope of the claims be limited to the specific values recited when defining a range. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

As used herein, the term “immunoglobulin” (Ig) is used interchangeably with “antibody” herein. The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

The terms “full-length antibody,” “intact antibody” or “whole antibody” are used interchangeably to refer to an antibody, such as an anti-Siglec-9 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding.

“Functional fragments” of antibodies, such as anti-Siglec-9 antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the VH and VL domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the VH and VL domains of the two antibodies are present on different polypeptide chains.

The terms “anti-Siglec-9 antibody,” “Siglec-9 antibody” and “antibody that binds to Siglec-9” refer to an antibody that is capable of binding human Siglec-9 (sialic acid binding Ig like lectin 9, also known as CD329; CDw329; FOAP-9; siglec-9; OBBP-LIKE) with sufficient affinity such that the antibody is useful as a diagnostic and/or therapeutic agent in targeting Siglec-9. The extent of binding of an anti-Siglec-9 antibody to an unrelated, non-Siglec-9 protein can be less than about 10% of the binding of the antibody to Siglec-9 as measured, e.g., by a radioimmunoassay (RIA).

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody. As used herein, the term “light chain” when used in reference to an antibody can refer to any distinct type, e.g., kappa (x) or lambda (k) based on the amino acid sequence of the constant domains. Light chain amino acid sequences are well known in the art. In specific embodiments, the light chain is a human light chain.

The terms “VH” and “VH domain” are used interchangeably to refer to the heavy chain variable region of an antibody. As used herein, the term “heavy chain” when used in reference to an antibody can refer to any distinct type, e.g., alpha (a), delta (6), epsilon (F), gamma (γ), and mu (p), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄. Heavy chain amino acid sequences are well known in the art. In specific embodiments, the heavy chain is a human heavy chain.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat EA & Wu TT (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR-H1), amino acid positions 50 to 65 (CDR-H2), and amino acid positions 95 to 102 (CDR-H3). Using the Kabat numbering system, CDRs within an antibody light chain molecule are typically present at amino acid positions 24 to 34 (CDR-L1), amino acid positions 50 to 56 (CDR-L2), and amino acid positions 89 to 97 (CDR-L3). In a specific embodiment, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

Kabat Numbering

| | | | |
|---|---|---|---|
| H1 | H31-H35 | H26-H35 | H26-H32 |

Chothia Numbering

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC), antibody-dependent cellular phagocytosis (ADCP), or complement-dependent cytotoxicity (CDC).

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some embodiments, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

An “affinity-matured” antibody, such as an anti-Siglec-9 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schieretal. Gene 169:147-155 (1995); Yeltonetal. J. Immunol. 155:1994-2004 (1995); Jackson et al., J. Immunol. 154(7):3310-9 (1995); and Hawkins et al., J. Mol. Biol. 226:889-896 (1992).

As used herein, an “interaction” between a Siglec-9 protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody or antigen-binding fragment thereof binds can be determined by, e.g., epitope mapping, NMR spectroscopy, X-ray diffraction crystallography studies, ELISA assays, surface plasmon resonance (SPR), biolayer interferometry (BLI), hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen NE (1997) Structure 5: 1269-1274; McPherson A (1976) J Biol Chem 251: 6300-6303). Antibody/antigen-binding fragment thereof: antigen crystals can be studied using well-known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al., U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter CW; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

A Siglec-9 antibody that “binds to the same epitope” as a reference Siglec-9 antibody refers to an antibody that binds to the same Siglec-9 amino acid residues as the reference Siglec-9 antibody. The ability of a Siglec-9 antibody to bind to the same epitope as a reference Siglec-9 antibody is determined by a hydrogen/deuterium exchange assay (see Coales et al. Rapid Commun. Mass Spectrom. 2009; 23: 639-647).

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen binding domain and the epitope. Accordingly, an antibody that “specifically binds” to human Siglec-9 (SEQ ID NO: 1) may also bind to Siglec-9 from other species (e.g., cynomolgous monkey, mouse, and/or rat Siglec-9) and/or Siglec-9 proteins produced from other human alleles, but the extent of binding to an un-related, non-Siglec-9 protein (e.g., other Siglec protein family members such as Siglec-7) is less than about 10% of the binding of the antibody to Siglec-9 as measured, e.g., by a radioimmunoassay (RIA).

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

A “blocking” antibody, an “antagonist” antibody, or an “inhibitory” antibody is an antibody, such as an anti-Siglec-9 antibody of the present disclosure, that inhibits or reduces (e.g., decreases) antigen binding to one or more ligands after the antibody binds the antigen, and/or that inhibits or reduces (e.g., decreases) one or more activities or functions of the antigen after the antibody binds the antigen. In some embodiments, blocking antibodies, antagonist antibodies, or inhibitory antibodies substantially or completely inhibit antigen binding to one or more ligand and/or one or more activities or functions of the antigen.

An “agonist” antibody or an “activating” antibody is an antibody, such as an agonist anti-Siglec-9 antibody of the present disclosure, that induces (e.g., increases) one or more activities or functions of the antigen after the antibody binds the antigen.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU or Kabat numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgGI Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

“Percent identity” and “homology” refer to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the BLASTN program set at default parameters, and alignment of amino acid sequences can be performed with the BLASTP program set at default parameters (see National Center for Biotechnology Information (NCBI) on the worldwide web, ncbi.nlm.nih.gov).

As used herein, the term “cell” can be any type of cell, e.g., a primary cell, a cell in culture, or a cell from a cell line that can be or has been a recipient for vector(s) for incorporation of polynucleotide inserts. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

The terms “administer”, “administering”, “administration”, and the like, as used herein, refer to methods that may be used to enable delivery of a drug, e.g., an anti-Siglec-9 antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

Administration “in combination with” one or more further therapeutic agents includes simultaneous (concurrent) or consecutive (sequential) administration in any order.

The combination therapy can provide “synergy,” i.e., the effect achieved when the active agents used together is greater than the sum of the effects that result from using the active agents separately. A synergistic effect can be attained when the active agents are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the active agents are administered or delivered sequentially, e.g., by different injections in separate syringes. A “synergistic combination” produces an effect that is greater than the sum of the effects of the individual active agents of the combination.

The combination therapy can provide an “additive” effect, i.e., the effect achieved when the active agents used together is equal to the sum of the effects the result from using the active agents separately.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a cynomolgus monkey. In some embodiments, the subject is a human.

The term “therapeutically effective amount” refers to an amount of a drug, e.g., an anti-Siglec-9 antibody or antigen-binding fragment thereof effective to treat a disease or disorder in a subject. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. To the extent the drug can prevent growth and/or kill existing cancer cells, it can be cytostatic and/or cytotoxic.

The terms “excipient” and “carrier” are used interchangeably and refer to an inert substance added to a pharmaceutical composition to further facilitate administration of a compound, e.g., a antibody or antigen-binding fragment, a conjugate, a CAR, or a cell engineered to express a CAR of the present disclosure.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the phrase “subject in need thereof” includes subjects, such as mammalian subjects, that would benefit from the administration of a composition described herein, e.g., to improve one or more symptoms associated with a disease or disorder described herein (e.g., cancer).

The terms “cancer” and “cancerous” refer to or describe the physiological condition in mammals in which a population of cells are characterized by unregulated cell growth. A “cancer” or “cancer tissue” can include a tumor at various stages.

The term “vector,” as used herein, is intended to refer to a nucleic acid molecule capable of transporting another nucleic acid to which it has been linked. One type of vector is a “plasmid,” which refers to a circular double-stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

As used herein, “cell engineering” or “cell modification” (including derivatives thereof) refers to the targeted modification of a cell, e.g., an immune cell disclosed herein. In some aspects, the cell engineering comprises viral genetic engineering, non-viral genetic engineering, introduction of receptors to allow for tumor-specific targeting (e.g., an anti-Siglec-9 CAR) introduction of one or more endogenous genes that improve T cell function, introduction of one or more synthetic genes that improve immune cell, e.g., T cell function, or any combination thereof. As further described elsewhere in the present disclosure, in some aspects, a cell can be engineered or modified with a transcription activator (e.g., CRISPR/Cas system-based transcription activator), wherein the transcription activator is capable of inducing and/or increasing the endogenous expression of a protein of interest.

The term “chimeric antigen receptor” or alternatively a “CAR” refers to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides includes a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (e.g., CD3 zeta). In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule.

In some aspects, the CAR comprises a chimeric fusion protein comprising an antigen-binding domain, a transmembrane domain, and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

While the present application often uses CARs to illustrate the different aspects of the disclosed subject matter, it will be apparent to a skilled artisan that the relevant disclosures provided herein can equally apply to other chimeric binding proteins. As used herein, the term “chimeric binding protein” refers to proteins that are capable of binding to one or more antigens (e.g., comprising an antigen-binding moiety) and are created through the joining of two or more heterologous polynucleotides which originally coded for separate proteins or fragments of proteins or multiple fragments of the same protein connected in a non-naturally occurring orientation. Non-limiting examples of other chimeric binding proteins include a T cell receptor (TCR) (e.g., engineered TCR), chimeric antibody-T cell receptor (caTCR), chimeric signaling receptor (CSR), T cell receptor mimic (TCR mimic), and combinations thereof. Accordingly, unless indicated otherwise, the term CARs, in some aspects, can encompass other types of chimeric binding proteins known in the art, e.g., those described herein.

As used herein, the term “reference CAR T cell” refers to a corresponding CAR T cell comprising the same structural CAR components but does not express Siglec-9 antibody or antigen-binding fragment.

The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain can generate a signal that promotes an immune effector function of the CAR-containing cell, e.g., an anti-Siglec-9 CAR T cell described herein. Non-limiting examples of immune effector function, e.g., in a CAR T cell, include cytolytic activity and helper activity, including the secretion of cytokines. In some aspects, the intracellular signal domain is the portion of the protein which transduces the effector function signal and directs the cell to perform a specialized function. While the entire intracellular signaling domain can be employed, in many cases, it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

### B. Siglec-9 Proteins

In one aspect, the present disclosure provides agents, such as isolated (e.g., monoclonal) antibodies, that interact with or otherwise bind to regions, such as epitopes, within a Siglec-9 protein of the present disclosure. In some embodiments, agents of the present disclosure, such as anti-Siglec-9 antibodies of the present disclosure, bind to a Siglec-9 protein and modulate one or more Siglec-9 activities after binding to the Siglec-9 protein, for example, an activity associated with Siglec-9 expression in a cell. Siglec-9 proteins of the present disclosure include, without limitation, a mammalian Siglec-9 protein, human Siglec-9 protein, mouse Siglec-9 protein, and rat Siglec-9 protein.

Siglec-9 is variously referred to as a Siglec-9 molecule, Sialic acid-binding Ig-like lectin 9, CD329 antigen, CD329; CDw329, FOAP-9, and OBBP-LIKE.

Siglec-9 is an immunoglobulin-like receptor primarily expressed on myeloid lineage cells, including without limitation, macrophages, neutrophils, dendritic cells, osteoclasts, monocytes, and microglia; lymphoid lineage cells, including without limitation NK cells and T cells; and tumor cells. In some embodiments, Siglec-9 forms a receptor-signaling complex with CD64. In some embodiments, Siglec-9 signaling results in the downstream inhibition of PI3K or other intracellular signals. On myeloid cells, Toll-like receptor (TLR) signals are important for the inhibition of Siglec-9 activities, e.g., in the context of an infection response. TLRs also play a key role in the pathological inflammatory response, e.g., TLRs expressed in macrophages, neutrophils, NK cells and dendritic cells.

Various Siglec-9 homologs are known, including without limitation, human Siglec-9, chimpanzee Siglec-9, green monkey Siglec-9, rhesus macaque Siglec-9, and mouse Siglec-9 (Siglec-E). The amino acid sequence of human Siglec-9 is set forth below and as SEQ ID NO: 1:

MLLLLLPLLWGRERAEGQTSKLLTMQSSVTVQEGLCVHVPCSFSYPSHGW
IYPGPVVHGYWFREGANTDQDAPVATNNPARAVWEETRDRFHLLGDPHTK
NCTLSIRDARRSDAGRYFFRMEKGSIKWNYKHHRLSVNVTALTHRPNILI
PGTLESGCPQNLTCSVPWACEQGTPPMISWIGTSVSPLDPSTTRSSVLTL
IPQPQDHGTSLTCQVTFPGASVTTNKTVHLNVSYPPQNLTMTVFQGDGTV
STVLGNGSSLSLPEGQSLRLVCAVDAVDSNPPARLSLSWRGLTLCPSQPS
NPGVLELPWVHLRDAAEFTCRAQNPLGSQQVYLNVSLQSKATSGVTQGVV
GGAGATALVFLSFCVIFVVVRSCRKKSARPAAGVGDTGIEDANAVRGSAS
QGPLTEPWAEDSPPDQPPPASARSSVGEGELQYASLSFQMVKPWDSRGQE
ATDTEYSEIKIHR.

In some embodiments, the Siglec-9 is a preprotein that includes a signal sequence. In some embodiments, the Siglec-9 is a mature protein. In some embodiments, the mature Siglec-9 protein does not include a signal sequence. In some embodiments, the mature Siglec-9 protein is expressed on a cell. In some embodiments, the mature Siglec-9 protein is expressed on a cell, such as the surface of a cell, including, without limitation, human dendritic cells, human macrophages, human monocytes, human osteoclasts, human neutrophils, human T cells, human helper T cell, human cytotoxic T cells, human granulocytes, and human microglia. Agents of the present disclosure, such as anti-Siglec-9 antibodies of the present disclosure, may bind any of the Siglec-9 proteins of the present disclosure expressed on any cell disclosed herein.

In some embodiments, Siglec-9 agents of the present disclosure, such as antagonist anti-Siglec-9 antibodies of the present disclosure, may increase the activity of cytotoxic T cells helper T cells or both. In some embodiments, Siglec-9 agents of the present disclosure, such as antagonist anti-Siglec-9 antibodies of the present disclosure, are beneficial for preventing, lowering the risk of, or treating conditions and/or diseases associated with decreased activity of cytotoxic T cells helper T cells or both, including without limitation, tumors, including solid tumors such as bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer.

In some embodiments, Siglec-9 agents of the present disclosure, such as agonist anti-Siglec-9 antibodies of the present disclosure, may decrease the activity of neutrophils. In some embodiments, Siglec-9 agents of the present disclosure, such as agonist anti-Siglec-9 antibodies of the present disclosure, are beneficial for preventing, lowering the risk of, or treating conditions and/or diseases associated with decreased activity of the activity of natural killer cells, neutrophils or both, including without limitation, tumors, including solid tumors such as bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer.

In some embodiments, Siglec-9 agents of the present disclosure, such as antagonist anti-Siglec-9 antibodies of the present disclosure, may increase the killing activity of NK cells. In some embodiments, Siglec-9 agents of the present disclosure, such as antagonistic anti-Siglec-9 antibodies of the present disclosure, are beneficial for preventing, lowering the risk of, or treating conditions and/or diseases associated with decreased activity of natural killer cells, neutrophils or both, including without limitation, tumors, including solid tumors such as bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer.

In some embodiments, Siglec-9 agents of the present disclosure, such as agonist anti-Siglec-9 antibodies of the present disclosure, may decrease the activity, decrease the proliferation, decrease the survival, decrease the functionality, decrease infiltration to tumors or lymphoid organs (e.g., the spleen and lymph nodes), and/or promote apoptosis of T-regulatory cells or inhibitory tumor-imbedded immunosuppressor dendritic cells or, tumor-associated macrophages, tumor-associated neutrophils, tumor-associated NK cells, or, myeloid-derived suppressor cells. In some embodiments, Siglec-9 agents of the present disclosure, such as agonist anti-Siglec-9 antibodies of the present disclosure, are beneficial for preventing, lowering the risk of, or treating conditions and/or diseases associated with the activity of one or more types of immune suppressor cells, including without limitation, tumors, including solid tumors that do not express Siglec-9 such as bladder cancer, brain cancer, breast cancer, colon cancer, rectal cancer, endometrial cancer, kidney cancer, renal cell cancer, renal pelvis cancer, lung cancer, melanoma, non-Hodgkin's lymphoma, pancreatic cancer, prostate cancer, ovarian cancer, fibrosarcoma, thyroid cancer, and blood tumors that express Siglec-9, such as leukemia cells.

Certain aspects of the present disclosure provide anti-Siglec-9 antibodies that bind to a human Siglec-9, or a homolog thereof, including without limitation a mammalian Siglec-9 protein and Siglec-9 orthologs from other species. Exemplary Siglec-9 homologs and orthologs include Chimpanzee (Pan troglodytes; NCBI Accession No. XP_003316614), Green monkey (Chlorocebus sabaeus; NCBI Accession No. XP_007995940.1), Rhesus macaque (Macaca mulatta; NCBI Accession No. XP_001114560.2), and Mouse (Mus musculus; NCBI Accession No. NP_112458.2).

In some embodiments, agents of the present disclosure that decrease cellular levels of Siglec-9 and/or inhibit interaction between Siglec-9 and one or more Siglec-9 ligands, or that bind or interact with Siglec-9, such as anti-Siglec-9 antibodies of the present disclosure, bind to a wild-type Siglec-9 protein of the present disclosure, naturally occurring variants thereof, and/or disease variants thereof.

In some embodiments, agents of the present disclosure that decrease cellular levels of Siglec-9 and/or inhibit interaction between Siglec-9 and one or more Siglec-9 ligands, or that bind or interact with Siglec-9, such as anti-Siglec-9 antibodies of the present disclosure, bind a variant of human Siglec-9.

In some embodiments, agents of the present disclosure that decrease cellular levels of Siglec-9 and/or inhibit interaction between Siglec-9 and one or more Siglec-9 ligands, or that bind or interact with Siglec-9, such as anti-Siglec-9 antibodies of the present disclosure, bind to a Siglec-9 protein expressed on the surface of a cell including, without limitation, human dendritic cells, human macrophages, human NK cells, human monocytes, human osteoclasts, human neutrophils, human T cells, human T helper cell, human cytotoxic T cells, human granulocytes, and human microglia. In some embodiments, agents of the present disclosure that decrease cellular levels of Siglec-9 and/or inhibit interaction between Siglec-9 and one or more Siglec-9 ligands, or that bind or interact with Siglec-9, such as anti-Siglec-9 antibodies of the present disclosure, bind to a Siglec-9 protein expressed on the surface of a cell and modulate (e.g., induce or inhibit) at least one Siglec-9 activity of the present disclosure after binding to the surface expressed Siglec-9 protein. In some embodiments of the present disclosure, the anti-Siglec-9 antibody binds specifically to a Siglec-9 protein. In some embodiments of the present disclosure, the anti-Siglec-9 antibody further binds to at least one additional Siglec protein. In some embodiments, the anti-Siglec-9 antibody modulates one or more activities of at least one additional Siglec protein or of a cell expressing the at least one additional Siglec protein.

Siglec-9 proteins of the present disclosure can interact with (e.g., bind to) one or more Siglec-9 ligands.

Exemplary Siglec-9 ligands include, without limitation, sialic acid, sialic acid-containing glycolipids, sialic acid-containing glycoproteins, alpha-2,8-disialyl containing glycolipids, branched alpha-2,6-linked sialic acid-containing glycoproteins, terminal alpha-2,6-linked sialic acid-containing glycolipids, terminal alpha-2,3-linked sialic acid-containing glycoproteins, disialogangliosides (e.g., gangliosides or glycolipids containing a ceramide linked to a sialylated glycan), secreted mucins, Siglec-9 ligands expressed on red blood cells, Siglec-9 ligands expressed on bacterial cells, Siglec-9 ligands expressed on apoptotic cells, Siglec-9 ligands expressed on nerve cells, Siglec-9 ligands expressed on glia cells, Siglec-9 ligands expressed on microglia, Siglec-9 ligands expressed on astrocytes, Siglec-9 ligands expressed on tumor cells, Siglec-9 ligands expressed on viruses, Siglec-9 ligands expressed on the receptor binding domain of SARS-CoV-2 Omicron variant and other variants, Siglec-9 ligands expressed on dendritic cells, Siglec-9 ligands bound to beta amyloid plaques, Siglec-9 ligands bound to Tau tangles, Siglec-9 ligands on disease-causing proteins, Siglec-9 ligands on disease-causing peptides, Siglec-9 ligands expressed on macrophages, Siglec-9 ligands expressed on neutrophils, Siglec-9 ligands expressed on natural killer cells, Siglec-9 ligands expressed on monocytes, Siglec-9 ligands expressed on T cells, Siglec-9 ligands expressed on T helper cells, Siglec-9 ligands expressed on cytotoxic T cells, Siglec-9 ligands expressed on B cells, Siglec-9 ligands expressed on tumor-imbedded immunosuppressor dendritic cells, Siglec-9 ligands expressed on tumor-imbedded immunosuppressor macrophages, Siglec-9 ligands expressed on myeloid-derived suppressor cells, Siglec-9 ligands expressed on regulatory T cells. In some embodiments, Siglec-9 ligands of the present disclosure are ganglioside (e.g., disialogangliosides). Disialogangliosides generally share a common lacto-ceramide core and one or more sialic acid residues. Additional Siglec-9 ligands include MFGM (Milk Fat Globule-EGF Factor 8 Protein, also known as Lactadherin), NUCB1 (Nucleobindin-1), LGALS3BP (Galectin-3-binding protein), NELL2 (Protein kinase C-binding protein NELL2), and APP (Amyloid-beta precursor protein).

### C. Anti-Siglec-9 Antibodies

Certain aspects of the present disclosure relate to anti-Siglec-9 antibodies that decrease cellular levels of Siglec-9 and/or inhibit interaction (e.g., binding) between Siglec-9 and one or more Siglec-9 ligands. In some embodiments, the anti-Siglec-9 antibody decreases cellular levels of Siglec-9 without inhibiting the interaction (e.g., binding) between Siglec-9 and one or more Siglec-9 ligands. In some embodiments, the anti-Siglec-9 antibody inhibits the interaction (e.g., binding) between Siglec-9 and one or more Siglec-9 ligands. In some embodiments, the anti-Siglec-9 antibody decreases cellular levels of Siglec-9 and inhibits the interaction (e.g., binding) between Siglec-9 and one or more Siglec-9 ligands. Other aspects of the present disclosure relate to anti-Siglec-9 antibodies that bind Siglec-9 without decreasing cellular levels of Siglec-9 and/or without inhibiting interaction (e.g., binding) between Siglec-9 and one or more Siglec-9 ligands.

Therefore, in some embodiments, antibodies of the present disclosure that bind a Siglec-9 protein may include agonist antibodies that due to their epitope specificity bind Siglec-9 and transiently activate one or more Siglec-9 activities before they, for example, decrease cellular levels of Siglec-9, inhibit one or more Siglec-9 activities (e.g., due to decreased cellular levels of Siglec-9), and/or inhibit interaction (e.g., binding) between Siglec-9 and one or more Siglec-9 ligands. In some embodiments, such antibodies may bind to the ligand-binding site on Siglec-9 and transiently mimic the action of a natural ligand. Alternatively, such antibodies may stimulate the target antigen to transduce a signal by binding to one or more domains that are not the ligand-binding sites. In some embodiments, such antibodies would not interfere with ligand binding. In some embodiments, regardless of whether antibodies bind or do not bind to the ligand-binding site on Siglec-9, the antibodies may subsequently act as longer-term inhibitors of Siglec-9 expression and/or one or more activities of a Siglec-9 protein by inducing Siglec-9 degradation, Siglec-9 desensitization, Siglec-9 cleavage, Siglec-9 internalization, Siglec-9 shedding, downregulation of Siglec-9 expression, and/or lysosomal degradation of Siglec-9.

In some embodiments, the anti-Siglec-9 antibodies inhibit interaction (e.g., binding) between a Siglec-9 protein of the present disclosure and one or more Siglec-9 ligands including, without limitation, Siglec-9 ligands expressed on red blood cells, Siglec-9 ligands expressed on bacterial cells, Siglec-9 ligands expressed on apoptotic cells, Siglec-9 ligands expressed on nerve cells, Siglec-9 ligands expressed on glia cells, Siglec-9 ligands expressed on microglia, Siglec-9 ligands expressed on astrocytes, Siglec-9 ligands expressed on tumor cells, Siglec-9 ligands expressed on viruses, Siglec-9 ligands expressed on dendritic cells, Siglec-9 ligands bound to beta amyloid plaques, Siglec-9 ligands bound to Tau tangles, Siglec-9 ligands on disease-causing proteins, Siglec-9 ligands on disease-causing peptides, Siglec-9 ligands expressed on macrophages, Siglec-9 ligands expressed on neutrophils, Siglec-9 ligands expressed on natural killer cells, Siglec-9 ligands expressed on monocytes, Siglec-9 ligands expressed on T cells, Siglec-9 ligands expressed on T helper cells, Siglec-9 ligands expressed on cytotoxic T cells, Siglec-9 ligands expressed on B cells, Siglec-9 ligands expressed on tumor-imbedded immunosuppressor dendritic cells, Siglec-9 ligands expressed on tumor-imbedded immunosuppressor macrophages, Siglec-9 ligands expressed on myeloid-derived suppressor cells, Siglec-9 ligands expressed on regulatory T cells, secreted mucins, sialic acid, sialic acid-containing glycolipids, sialic acid-containing glycoproteins, alpha-2,8-disialyl containing glycolipids, branched alpha-2,6-linked sialic acid-containing glycoproteins, terminal alpha-2,6-linked sialic acid-containing glycolipids, terminal alpha-2,3-linked sialic acid-containing glycoproteins, and gangliosides (e.g., disialogangliosides), novel Siglec-9 ligands: MFGM (Milk Fat Globule-EGF Factor 8 Protein, also known as Lactadherin), NUCB1 (Nucleobindin-1), LGALS3BP (Galectin-3-binding protein), NELL2 (Protein kinase C-binding protein NELL2), and APP (Amyloid-beta precursor protein).

In some embodiments, anti-Siglec-9 antibodies of the present disclosure bind to a Siglec-9 protein of the present disclosure expressed on the surface of a cell and the naked antibodies inhibit interaction (e.g., binding) between the Siglec-9 protein and one or more Siglec-9 ligands. In some embodiments, anti-Siglec-9 antibodies of the present disclosure that bind to a Siglec-9 protein of the present disclosure inhibit interaction (e.g., binding) between the Siglec-9 protein and one or more Siglec-9 ligands by reducing the effective levels of Siglec-9 that is available to interact with these proteins either on the cell surface or inside the cell. In some embodiments, anti-Siglec-9 antibodies of the present disclosure that bind to a Siglec-9 protein of the present disclosure inhibit interaction (e.g., binding) between the Siglec-9 protein and one or more Siglec-9 ligands by inducing degradation of Siglec-9.

In some embodiments that may be combined with any of the preceding embodiments, the anti-Siglec-9 antibody exhibits one or more activities selected from the group consisting of (a) increasing the number and/or the cytotoxic capacity of tumor infiltrating CD3+ T cells; (b) decreasing cellular levels of Siglec-9 in non-tumorigenic CD14+ myeloid cells, optionally wherein the non-tumorigenic CD14+ myeloid cells are tumor infiltrating cells or optionally wherein the non-tumorigenic CD14+ myeloid cells are present in blood; (c) reducing the number of non-tumorigenic CD14+ myeloid cells, optionally wherein the non-tumorigenic CD14+ myeloid cells are tumor infiltrating cells or optionally wherein the non-tumorigenic CD14+ myeloid cells are present in blood; (d) reducing PD-L1 levels in one or more cells, optionally wherein the one or more cells are non-tumorigenic myeloid-derived suppressor cells (MDSC); (e) reducing PD-L2 levels in one or more cells, optionally wherein the one or more cells are non-tumorigenic myeloid-derived suppressor cells (MDSC); (f) reducing B7-H2 levels in one or more cells, optionally wherein the one or more cells are non-tumorigenic myeloid-derived suppressor cells (MDSC); (g) reducing B7-H3 levels in one or more cells, optionally wherein the one or more cells are non-tumorigenic myeloid-derived suppressor cells (MDSC); (h) reducing CD200R levels in one or more cells, optionally wherein the one or more cells are non-tumorigenic myeloid-derived suppressor cells (MDSC); (i) reducing CD163 levels in one or more cells, optionally wherein the one or more cells are non-tumorigenic myeloid-derived suppressor cells (MDSC); (j) reducing CD206 levels in one or more cells, optionally wherein the one or more cells are non-tumorigenic myeloid-derived suppressor cells (MDSC); (k) decreasing tumor growth rate of solid tumors; (1) reducing tumor volume; (m) increasing efficacy of one or more PD-1 inhibitors; (n) increasing efficacy of one or more checkpoint inhibitor therapies and/or immune-modulating therapies, optionally wherein the one or more checkpoint inhibitor therapies and/or immune-modulating therapies target one or more of CTLA4, the adenosine pathway, PD-L1, PD-L2, PD-L1, PD-L2, OX40, TIM3, LAG3, or any combination thereof, (o) increasing efficacy of one or more chemotherapy agents, optionally wherein the one or more of the chemotherapy agents are gemcitabine, capecitabine, anthracyclines, doxorubicin (Adriamycin®), epirubicin (Ellence®), taxanes, paclitaxel (Taxol®), docetaxel (Taxotere®), 5-fluorouracil (5-FU), cyclophosphamide (Cytoxan®), carboplatin (Paraplatin®), and any combination thereof, (p) increasing proliferation of T cells in the presence of non-tumorigenic myeloid-derived suppressor cells (MDSC); (q) inhibiting differentiation, survival, and/or one or more functions of non-tumorigenic myeloid-derived suppressor cells (MDSC); (r) killing Siglec-9-expressing immunosuppressor non-tumorigenic myeloid cells and/or non-tumorigenic CD14-expressing cells in solid tumors and associated blood vessels when conjugated to a chemical or radioactive toxin. In some embodiments that may be combined with any of the preceding embodiments, the anti-Siglec-9 antibody is not conjugated to an agent, optionally wherein the agent is drug, toxin, chemotherapeutic, or radioisotope; (s) inducing Antibody-Dependent Cellular Cytotoxicity (ADCC), Antibody-Dependent Cellular Phagocytosis (ADCP), or Complement-Dependent Cytotoxicity (CDC); (t) engaging immune effector cells to mediate the destruction of Siglec-9 expressing cells; (u) improving the ability of dendritic cells to present antigens; (v) enhancing the activation of T cells; (w) modulating tumor microenvironment acidity; (x) improving oxygen delivery to the tumor; and (y) enhancing the efficacy of radiation therapy and chemotherapeutic agents.

In some embodiments, anti-Siglec-9 antibodies of the present disclosure are agonist antibodies or antagonist antibodies that bind to a Siglec-9 protein of the present disclosure expressed on the surface of a cell and modulate (e.g., induce or inhibit) one or more Siglec-9 activities of the present disclosure after binding to the surface-expressed Siglec-9 protein. In some embodiments, anti-Siglec-9 antibodies of the present disclosure are inert antibodies.

In certain embodiments, an antibody or antigen-binding fragment thereof described herein binds to Siglec-9.

In certain embodiments, an antibody or antigen-binding fragment thereof described herein binds to Siglec-9 and comprises the six CDRs of an antibody listed in Tables 1 and 2 (i.e., the three Variable Heavy (VH) CDRs of the antibody listed in Table 1 and the three Variable Light (VL) CDRs of the same antibody listed in Table 2).

TABLE 1
Variable Heavy (VH) CDR Amino Acid Sequences

| Antibody | CDR-H1 | CDR-H2 | CDR-H3 |
|---|---|---|---|
| ED2D5 | DYYMY (SEQ | TISDGGSDTYY | DPDYDDGRFAY |
| | ID NO: 5) | PDNMKG (SEQ | (SEQ ID NO: |
| | | ID NO: 6) | 7) |
| CB6B3 | DYFIH (SEQ | LIDPENDNTMY | TVVARSFDY |
| | ID NO: 13) | DPRFQV (SEQ | (SEQ ID NO: |
| | | ID NO: 14) | 15) |

TABLE 2
Variable Light (VL) CDR Amino Acid Sequences

| Antibody | CDR-L1 | CDR-L2 | CDR-L3 |
|---|---|---|---|
| ED2D5 | KASQDVGTVVA | WSSTRHT | QQYIDYPLS |
| | (SEQ ID NO: | (SEQ ID | (SEQ ID |
| | 8) | NO: 9) | NO: 10) |
| CB6B3 | KASQSVSNDVA | YTSNRYT | QQDFRSPFT |
| | (SEQ ID NO: | (SEQ ID | (SEQ ID |
| | 16) | NO: 17) | NO: 18) |

In certain embodiments, an antibody or antigen-binding fragment thereof described herein binds to Siglec-9 and comprises the VH of an antibody listed in Table 3.

TABLE 3
Variable Heavy Chain (VH) Amino Acid Sequences

| Antibody | VH Amino Acid Sequence |
|---|---|
| ED2D5 | EVQLVESGGGLVKPGGSLKLSCAASGFTFSDYYMYWVR |
| | QTPEKRLEWVATISDGGSDTYYPDNMKGRFTISRDNAK |
| | NNLYLQMSSLKSEDTAMYYCTRDPDYDDGRFAYWGQGT |
| | LVTVSA (SEQ ID NO: 3) |
| CB6B3 | EVQLQQSGAEVVRPGALVKLSCKAAGFNIKDYFIHWVK |
| | QRPEQGLEWIGLIDPENDNTMYDPRFQVKASLTADTSS |
| | NTAYLQLSRLTSEDAAVYYCASTVVARSFDYWGQGTTL |
| | TVSS (SEQ ID NO: 11) |
| humanised | EVQLVQSGAEVKKPGATVKISCKVSGYTFTDYFIHWVQ |
| CB6B3 | QAPGKGLEWMGLIDPENDNTMYDPRFQVRVTITADTST |
| | DTAYMELSSLRSEDTAVYYCASTVVARSFDYWGQGTTV |
| | TVSS (SEQ ID NO: 19) |

In certain embodiments, an antibody or antigen-binding fragment thereof described herein binds to Siglec-9 and comprises the VL of an antibody listed in Table 4.

TABLE 4
Variable Light Chain (VL) Amino Acid Sequences

| Antibody | VL Amino Acid Sequence |
|---|---|
| ED2D5 | DIVMTQSHKFMSTSVGDRVSITCKASQDVGTVVAWYQQ |
| | KSGQSPKLLIYWSSTRHTGVPDRFTGSGSGTDFTLTIS |
| | NVQSEDLADYFCQQYIDYPLSFGAGTKLELK (SEQ |
| | ID NO: 4) |
| CB6B3 | SIVMTQTPKFLLVSAGDRVTITCKASQSVSNDVAWYQQ |
| | KSGQSPKLLIYYTSNRYTGVPDRFTGSGYGTDFTFTIS |
| | TVQAEDLAVYFCQQDFRSPFTFGAGTKLELK (SEQ |
| | ID NO: 12) |
| humanised | AIQMTQSPSSLSASVGDRVTITCKASQSVSNDVAWYQQ |
| CB6B3 | KPGKAPKLLIYYTSNRYTGVPSRFSGSGSGTDFTLTIS |
| | SLQPEDFATYYCQQDFRSPFTFGGGTKVEIK (SEQ |
| | ID NO: 20) |

In certain embodiments, an antibody or antigen-binding fragment thereof described herein binds to Siglec-9 and comprises the VH and the VL of an antibody listed in Tables 3 and 4 (i.e., the VH of the antibody listed in Table 3 and the VL of the same antibody listed in Table 4).

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 80% identical to the VH sequence of the same antibody in Table 3 and a VL comprising a sequence at least 80% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 85% identical to the VH sequence of the same antibody in Table 3 and a VL comprising a sequence at least 85% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 90% identical to the VH sequence of the same antibody in Table 3 and a VL comprising a sequence at least 90% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 95% identical to the VH sequence of the same antibody in Table 3 and a VL comprising a sequence at least 95% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 96% identical to the VH sequence of the same antibody in Table 3 and a VL comprising a sequence at least 96% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 97% identical to the VH sequence of the same antibody in Table 3 and a VL comprising a sequence at least 97% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 98% identical to the VH sequence of the same antibody in Table 3 and a VL comprising a sequence at least 98% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 99% identical to the VH sequence of the same antibody in Table 3 and a VL comprising a sequence at least 99% identical to the VL sequence of the same antibody in Table 4.

In certain embodiments, an antibody or antigen-binding fragment thereof described herein binds to Siglec-9, comprises the six CDRs of an antibody listed in Tables 1 and 2 (i.e., the three VH CDRs of the antibody listed in Table 1 and the three VL CDRs of the same antibody listed in Table 2), and comprises the VH sequence of the same antibody in Table 3 and the VL sequence of the same antibody in Table 4.

In specific aspects, provided herein are antibodies that comprise a heavy chain and a light chain. With respect to the heavy chain, in a specific embodiment, the heavy chain of an antibody described herein can be an alpha (a), delta (6), epsilon (F), gamma (γ) or mu (p) heavy chain. In another specific embodiment, the heavy chain of an antibody described can comprise a human alpha (a), delta (6), epsilon (F), gamma (γ) or mu (p) heavy chain. In a particular embodiment, an antibody described herein, which immunospecifically binds to Siglec-9, comprises a heavy chain wherein the amino acid sequence of the VH domain comprises an amino acid sequence set forth in Table 3 and wherein the constant region of the heavy chain comprises the amino acid sequence of a human gamma (γ) heavy chain constant region. In a specific embodiment, an antibody described herein, which specifically binds to Siglec-9, comprises a heavy chain wherein the amino acid sequence of the VH domain comprises a sequence set forth in Table 3, and wherein the constant region of the heavy chain comprises the amino acid of a human heavy chain described herein or known in the art. Non-limiting examples of human constant region sequences have been described in the art, e.g., see U.S. Pat. No. 5,693,780 and Kabat E A et al., (1991) supra.

In another specific embodiment, the light chain of an antibody described herein is a lambda light chain. In yet another specific embodiment, the light chain of an antibody described herein is a human kappa light chain or a human lambda light chain. In a particular embodiment, an antibody described herein, which immunospecifically binds to a Siglec-9 polypeptide comprises a light chain wherein the amino acid sequence of the VL domain comprises a sequence set forth in Table 4, and wherein the constant region of the light chain comprises the amino acid sequence of a human kappa light chain constant region. In another particular embodiment, an antibody described herein, which immunospecifically binds to Siglec-9 comprises a light chain wherein the amino acid sequence of the VL domain comprises a sequence set forth in Table 4 and wherein the constant region of the light chain comprises the amino acid sequence of a human lambda light chain constant region. In a specific embodiment, an antibody described herein, which immunospecifically binds to Siglec-9 comprises a light chain wherein the amino acid sequence of the VL domain comprises a sequence set forth in Table 4 and wherein the constant region of the light chain comprises the amino acid sequence of a human kappa or lambda light chain constant region. Non-limiting examples of human constant region sequences have been described in the art, e.g., see U.S. Pat. No. 5,693,780 and Kabat E A et al., (1991) supra.

In a specific embodiment, an antibody described herein, which immunospecifically binds to Siglec-9 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In another specific embodiment, an antibody described herein, which immunospecifically binds to Siglec-9 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In a particular embodiment, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

The constant region of a human IgG1 heavy chain can comprise the following amino acid sequence:

(SEQ ID NO: 21)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

Non-limiting examples of human constant regions are described in the art, e.g., see Kabat E A et al., (1991) supra.

In certain embodiments, one, two, or more mutations (e.g., amino acid substitutions) are introduced into the Fc region of an antibody or antigen-binding fragment thereof described herein (e.g., CH2 domain (residues 231-340 of human IgG₁) and/or CH3 domain (residues 341-447 of human IgG₁) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity. In certain embodiments, mutations in the Fc region of an antibody or antigen-binding fragment thereof function to silence the Fc region (i.e., remove Fc function). These mutations may include NA (N297/A/Q/G), AAA (L235A/G237A/E318A), LALA (L234A/L235A), IgG4-PE (S228P/L235E), RR (G236R/L328R), GA (S298G/T299A), FES (L234F/L235E/P331S), IgG2m4 (H268Q/V309L/A330S/P331S), XmAb® bispecific (E233P/L234V/L235A/G236del/S267K), LALA-PG (L234A/L235A/P329G), IgG2c4d (V234A/G237A/P238S/H268A/V309L/A330S/P331S), or FEA mutations (L234F/L235E/D265A). In certain embodiments, the Fc region is modified to have reduced fucosylation.

In certain embodiments, any of the constant region mutations or modifications described herein can be introduced into one or both heavy chain constant regions of an antibody or antigen-binding fragment thereof described herein having two heavy chain constant regions.

In some embodiments, anti-Siglec-9 antibodies of the present disclosure may bind a conformational epitope. In some embodiments, anti-Siglec-9 antibodies of the present disclosure may bind a discontinuous Siglec-9 epitope. In some embodiments, the discontinuous Siglec-9 epitope may have two or more peptides, three or more peptides, four or more peptides, five or more peptides, six or more peptides, seven or more peptides, eight or more peptides, nine or more peptides, or 10 or more peptides. As disclosed herein, Siglec-9 epitopes may comprise one or more peptides comprising five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues of the amino acid sequence of SEQ ID NO: 1, or five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues on a mammalian Siglec-9 protein corresponding to the amino acid sequence of SEQ ID NO: 1.

### D. Antibody Production

Antibodies and antigen-binding fragments thereof that immunospecifically bind to Siglec-9 can be produced by any method known in the art for the synthesis of antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

Anti-Siglec-9 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, heteroconjugate antibodies, conjugated antibodies, library derived antibodies, antibodies having modified effector functions, fusion proteins containing an antibody portion, and any other modified configuration of the immunoglobulin molecule that includes an antigen recognition site, such as an epitope having amino acid residues of a Siglec-9 protein of the present disclosure, including glycosylation variants of antibodies, amino acid sequence variants of antibodies, and covalently modified antibodies. The anti-Siglec-9 antibodies may be human, murine, rat, or of any other origin (including chimeric or humanized antibodies).

### Polyclonal Antibodies

Polyclonal antibodies, such as polyclonal anti-Siglec-9 antibodies, are generally raised in animals by multiple subcutaneous (sc) or intraperitoneal (ip) injections of the relevant antigen and an adjuvant. It may be useful to conjugate the relevant antigen (e.g., a purified or recombinant Siglec-9 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 g (for rabbits) or 5 g (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies, such as monoclonal anti-Siglec-9 antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal anti-Siglec-9 antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant Siglec-9 protein of the present disclosure). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., a purified or recombinant Siglec-9 protein of the present disclosure) or a fusion variant thereof. Generally, peripheral blood lymphocytes (“PBLs”) are used if cells of human origin are desired, while spleen or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, Calif USA), as well as SP-2 cells and derivatives thereof (e.g., X63-Ag8-653) (available from the American Type Culture Collection, Manassas, Va. USA). Human myeloma and mouse-human heteromyeloma cell lines have also been described for the production of human monoclonal antibodies (Kozbor, J Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., a Siglec-9 protein of the present disclosure). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., a Siglec-9 protein of the present disclosure). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-Siglec-9 monoclonal antibodies may also be made by recombinant DNA methods, such as those disclosed in U.S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opin. Immunol., 5:256-262 (1993) and Plückthun, Immunol. Rev. 130:151-188 (1992).

In certain embodiments, anti-Siglec-9 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind a Siglec-9 protein of the present disclosure).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., anti-Siglec-9 antibodies of the present disclosure or fragments thereof) may be monovalent, the preparation of which is well-known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid anti-Siglec-9 antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Humanized Antibodies

Anti-Siglec-9 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human anti-Siglec-9 antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., Siglec-9 proteins of the present disclosure), is achieved. In general, the CDR residues are directly and most substantially involved in influencing antigen binding.

Various forms of the humanized anti-Siglec-9 antibody are contemplated. For example, the humanized anti-Siglec-9 antibody may be an antibody fragment, such as an Fab, which is optionally conjugated with one or more cytotoxic agent(s) in order to generate an immunoconjugate. Alternatively, the humanized anti-Siglec-9 antibody may be an intact antibody, such as an intact IgG1 antibody.

### Human Antibodies

Alternatively, human anti-Siglec-9 antibodies can be generated. For example, it is now possible to produce transgenic animals (e.g., mice) that are capable, upon immunization, of producing a full repertoire of human antibodies in the absence of endogenous immunoglobulin production. The homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Nat'l Acad. Sci. USA, 90:2551 (1993); Jakobovits et al., Nature, 362:255-258 (1993); Bruggermann et al., Year in Immunol., 7:33 (1993); U.S. Pat. No. 5,591,669 and WO 97/17852.

Alternatively, phage display technology can be used to produce human anti-Siglec-9 antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also U.S. Pat. Nos. 5,565,332 and 5,573,905. Additionally, yeast display technology can be used to produce human anti-Siglec-9 antibodies and antibody fragments in vitro (e.g., WO 2009/036379; WO 2010/105256; WO 2012/009568; US 2009/0181855; US 2010/0056386; and Feldhaus and Siegel (2004) J. Immunological Methods 290:69-80). In other embodiments, ribosome display technology can be used to produce human anti-Siglec-9 antibodies and antibody fragments in vitro (e.g., Roberts and Szostak (1997) Proc Natl Acad Sci 94:12297-12302; Schaffitzel et al. (1999) J. Immunolical Methods 231:119-135; Lipovsek and Plückthun (2004) J. Immunological Methods 290:51-67).

The techniques of Cole et al., and Boerner et al., are also available for the preparation of human anti-Siglec-9 monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human anti-Siglec-9 antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human anti-Siglec-9 antibodies may also be generated in vitro by activated B-cells (see U.S. Pat. Nos. 5,567,610 and 5,229,275).

### Antibody Fragments

In certain embodiments there are advantages to using anti-Siglec-9 antibody fragments, rather than whole anti-Siglec-9 antibodies. Smaller fragment sizes allow for rapid clearance and better brain penetration. Certain aspects of the present disclosure relate to antibody fragments that bind to one or more of a Siglec-9 protein of the present disclosure, a naturally occurring variant of a Siglec-9 protein, and a disease variant of a Siglec-9 protein. In some embodiments, the antibody fragment is an Fab, Fab′, Fab′-SH, F(ab′)2, Fv or scFv fragment.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host cells, for example, using nucleic acids encoding anti-Siglec-9 antibodies of the present disclosure. Fab, Fv and scFv antibody fragments can all be expressed in and secreted from E. coli, thus allowing the straightforward production of large amounts of these fragments. A anti-Siglec-9 antibody fragments can also be isolated from the antibody phage libraries as discussed above. Alternatively, Fab′-SH fragments can be directly recovered from E. coli and chemically coupled to form F(ab′)2 fragments (Carter et al., Bio Technology 10:163-167 (1992)). According to another approach, F(ab′)₂ fragments can be isolated directly from recombinant host cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-Siglec-9 antibody fragment may also be a “linear antibody,” e.g., as described in U.S. Pat. No. 5,641,870. Such linear antibody fragments may be monospecific or bispecific.

In some embodiments, the antibody fragment is used in combination with a second Siglec-9 antibody and/or with one or more antibodies that specifically bind a disease-causing protein selected from: amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein Al, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and any combination thereof, or with one or more antibodies that bind an immunomodulatory protein selected from: PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIM1, TIM3, TIM4, A2AR, LAG3, DR-5, CD2, CD3, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof.

In some embodiments, antibody fragments of the present disclosure may be functional fragments that bind the same epitope as any of the anti-Siglec-9 antibodies of the present disclosure. In some embodiments, the antibody fragments are miniaturized versions of the anti-Siglec-9 antibodies or antibody fragments of the present disclosure that have the same epitope of the corresponding full-length antibody, but have much smaller molecule weight. Such miniaturized anti-Siglec-9 antibody fragments may have better brain penetration ability and a shorter half-life, which is advantageous for imaging and diagnostic utilities (see e.g., Lutje S et al., Bioconjug Chem. 2014 Feb. 19; 25(2):335-41; Tavare R et al., Proc Natl Acad Sci USA. 2014 Jan. 21; 111(3):1108-13; and Wiehr S et al., Prostate. 2014 May; 74(7):743-55). Accordingly, in some embodiments, anti-Siglec-9 antibody fragments of the present disclosure have better brain penetration as compared to their corresponding full-length antibodies and/or have a shorter half-life as compared to their corresponding full-length antibodies.

### Bispecific and Polyspecific Antibodies

Bispecific antibodies (BsAbs) are antibodies that have binding specificities for at least two different epitopes, including those on the same or another protein (e.g., one or more Siglec-9 proteins of the present disclosure). Alternatively, one part of a BsAb can be armed to bind to the target Siglec-9 antigen, and another can be combined with an arm that binds to a second protein. Such antibodies can be derived from full-length antibodies or antibody fragments (e.g., F(ab′)₂ bispecific antibodies).

Methods for making bispecific antibodies are known in the art. Traditional production of full-length bispecific antibodies is based on the coexpression of two immunoglobulin heavy-chain/light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details on generating bispecific antibodies, see, for example, Suresh et al., Methods in Enzymology 121: 210 (1986).

According to another approach described in WO 96/27011 or U.S. Pat. No. 5,731,168, the interface between a pair of antibody molecules can be engineered to maximize the percentage of heterodimers which are recovered from recombinant cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell cultures have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al., J Immunol. 147:60 (1991).

Exemplary bispecific antibodies may bind to two different epitopes on a given molecule (e.g., a Siglec-9 protein of the present disclosure). Alternatively, an arm targeting a Siglec-9 signaling component may be combined with an arm which binds to a triggering molecule on a leukocyte such as a T cell receptor molecule (e.g., CD2, CD3, CD28 or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies may be bispecific T-cell engagers (BiTEs), which act through the simultaneous engagement of tumor-associated antigens (e.g., a Siglec-9 protein of the present disclosure) and a T cell receptor molecule (e.g., CD3), thereby resulting in T-cell mediated cytotoxicity specifically directed towards the tumor cells while also enhancing anti-tumor immunity. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF).

In some embodiments, bispecific antibodies of the present disclosure bind to one or more amino acid residues of a Siglec-9 protein of the present disclosure, such as one or more amino acid residues of human Siglec-9 (SEQ ID NO: 1), or amino acid residues on a Siglec-9 protein corresponding to amino acid residues of SEQ ID NO: 1. In some embodiments, bispecific antibodies of the present disclosure recognize a first antigen and a second antigen. In some embodiments, the first antigen is a Siglec-9 protein or a naturally occurring variant thereof. In some embodiments, the second antigen is also a Siglec-9 protein, or a naturally occurring variant thereof. In some embodiments, the second antigen is an antigen facilitating transport across the blood-brain-barrier (see, e.g., Gabathuler R., Neurobiol. Dis. 37 (2010) 48-57). Such second antigens include, without limitation, transferrin receptor (TR), insulin receptor (HIR), insulin-like growth factor receptor (IGFR), low-density lipoprotein receptor-related proteins 1 and 2 (LPR-1 and 2), diphtheria toxin receptor, CRM 197, a llama single domain antibody, TMEM 30(A), a protein transduction domain, TAT, Syn-B, penetratin, a poly-arginine peptide, Angiopep peptides such as ANG1005 (see, e.g., Gabathuler, 2010), and other cell surface proteins that are enriched on blood-brain barrier endothelial cells (see, e.g., Daneman et al., PLoS One. 2010 Oct. 29; 5(10):e13741). In some embodiments, the second antigen is a disease-causing protein including, without limitation, amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein Al, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides. In some embodiments, the second antigen is one or more ligands and/or proteins expressed on immune cells, including but not limited to PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIM1, TIM3, TIM4, A2AR, LAG3, DR-5, CD2, CD3, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof. In some embodiments, the second antigen is a protein, lipid, polysaccharide, or glycolipid expressed on one or more tumor cells. In some embodiments, bispecific antibodies of the present disclosure are biparatopic bispecific antibodies that simultaneously target distinct epitopes on Siglec-9, optimizing both specificity and affinity for Siglec-9 expressing cells, thereby enhancing the therapeutic potential through improved immune system modulation and target engagement.

### Multivalent Antibodies

A multivalent antibody may be internalized (and/or catabolized) faster than a bivalent antibody by a cell expressing an antigen to which the antibodies bind. The anti-Siglec-9 antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain. The multivalent antibodies may recognize the Siglec-9 antigen as well as, without limitation, additional antigens A beta peptide, antigen or an alpha synuclain protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), insulin receptor, insulin like growth factor receptor, transferrin receptor, or any other antigen that facilitates antibody transfer across the blood brain barrier.

### CAR-T Cells Engineered Against Siglec-9

CAR-T cells are engineered to target a range of antigens known for their roles in various cancers. These targets include, but are not limited to, CD19, CD20, CD22, CD30, BCMA (B-cell maturation antigen), and HER2. These antigens are selected based on their expression across a variety of hematological malignancies and solid tumors, making them prime candidates for CAR-T cell therapy. To amplify the therapeutic potential of these CAR-T cells, the present disclosure incorporates a novel strategy wherein these engineered T cells are also designed to secrete anti-Siglec-9 antibodies or their fragments. This dual-functional approach not only enables the CAR-T cells to directly engage and eliminate cancer cells via their CAR-mediated specificity but also modulates the tumor microenvironment through the secreted anti-Siglec-9 antibodies. The secretion of anti-Siglec-9 antibodies by CAR-T cells represents an innovative strategy to counteract immune suppression within the tumor milieu. By targeting Siglec-9, these antibodies can alleviate the immunosuppressive signals mediated by Siglec-9 interactions, enhancing the infiltration, persistence, and effector functions of not only the infused CAR-T cells but also the endogenous immune cells within the tumor. This dual mechanism overcomes the limitations faced by conventional CAR-T cell therapies and other immunotherapeutic approaches.

### Antibody Conjugates

Anti-Siglec-9 antibodies of the present disclosure, or antibody fragments thereof, can be conjugated to a detectable marker, a toxin, or a therapeutic agent. Any suitable method known in the art for conjugating molecules, such as a detectable marker, a toxin, or a therapeutic agent to antibodies may be used.

For example, drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Techniques to conjugate antibodies are disclosed are known in the art (see, e.g., Jane de Lartigue, OncLive Jul. 5, 2012; ADC Review on antibody-drug conjugates; and Ducry et al., (2010). Bioconjugate Chemistry 21 (1): 5-13).

An anti-Siglec-9 antibody or antigen-binding fragment thereof can be fused or conjugated (e.g., covalently or noncovalently linked) to a detectable label or substance. Examples of detectable labels or substances include enzyme labels, such as glucose oxidase; radioisotopes, such as iodine (125I, 121I), carbon (14C), sulfur (35S), tritium (3H), indium (1211n), and technetium (99Tc); luminescent labels, such as luminol; and fluorescent labels, such as fluorescein and rhodamine, and biotin. Such labeled antibodies or antigen-binding fragments thereof can be used to detect Siglec-9 (e.g., human Siglec-9) protein.

In some embodiments, the anti-Siglec-9 antibody or antigen-binding fragment thereof can be coupled to a payload. In some embodiments, the payload includes but is not limited to microtubule inhibitors (e.g., DM1, DM4, MMAE, MMAF), DNA damaging agents (e.g., calicheamicins, duocarmycins, PBD Dimers), topoisomerase inhibitors (e.g., SN-38, etoposide phosphate, exatecan, deruxtecan), RNA polymerase inhibitors (a-Amanitin), alkylating agents (bendamustine), immunomodulatory agents (e.g., immunostimulatory oligonucleotides, TLR agonists), proteasome inhibitors (e.g., bortezomib), anti-angiogenic agents (e.g., anecortave), RAD51 inhibitors (e.g., B02), and enzyme inhibitors (e.g., arginine deiminase), for broad-spectrum cancer targeting.

### E. Polynucleotides. Vectors, and Host Cells

For recombinant production of an anti-Siglec-9 antibody of the present disclosure, a polynucleotide encoding the anti-Siglec-9 antibody is isolated and inserted into one or more vectors for further cloning and/or expression in a host cell.

### Polynucleotides

Polynucleotides encoding an anti-Siglec-9 antibody or antigen-binding fragment thereof can be optimized, e.g., by codon/RNA optimization, replacement with heterologous signal sequences, and elimination of mRNA instability elements. Methods to generate optimized nucleic acids encoding an anti-Siglec-9 antibody or antigen-binding fragment thereof or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

A polynucleotide encoding an antibody or antigen-binding fragment thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In certain embodiments, the polynucleotide is a cDNA or a DNA lacking one more endogenous introns. In certain embodiments, a polynucleotide is a non-naturally occurring polynucleotide. In certain embodiments, a polynucleotide is recombinantly produced. In certain embodiments, the polynucleotides are isolated. In certain embodiments, the polynucleotides are substantially pure. In certain embodiments, a polynucleotide is purified from natural components.

### Vectors

Once a polynucleotide encoding an antibody or antigen-binding fragment thereof or domain thereof (e.g., heavy or light chain variable domain) described herein has been obtained, the vector for the production of the antibody or antigen-binding fragment thereof can be produced by recombinant DNA technology using techniques well known in the art.

Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColEl, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid. The expression vector may be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-Siglec-9 antibody of the present disclosure.

### Host Cells

An expression vector can be transferred to a cell (e.g., host cell) by conventional techniques and the resulting cells can then be cultured by conventional techniques to produce an antibody or antigen-binding fragment thereof described herein or a domain thereof. Thus, provided herein are host cells containing a polynucleotide encoding an antibody or antigen-binding fragment thereof described herein or a domain thereof, operably linked to a promoter for expression of such sequences in the host cell.

Suitable host cells for cloning or expression of antibody-encoding vectors include prokaryotic or eukaryotic cells. For example, anti-Siglec-9 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for the transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants.).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, anti-Siglec-9 antibodies described herein are produced in mammalian cells, such as CHO cells.

Once an antibody or antigen-binding fragment thereof described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies or antigen-binding fragments thereof described herein can be fused to heterologous polypeptide sequences described herein or otherwise known in the art to facilitate purification.

In specific embodiments, an antibody or antigen-binding fragment thereof described herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in a particular embodiment, a preparation of an antibody or antigen-binding fragment thereof described herein is substantially free of cellular material and/or chemical precursors.

### F. Chimeric Antigen Receptors

Among the provided binding molecules (e.g., anti-Siglec-9 antibodies) are recombinant receptors, such as chimeric antigen receptors (CARs). Also provided are cells expressing the CARs and uses thereof. Exemplary CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, e.g., and in which the antigen-binding portion, e.g., scFv, is replaced by an antibody or an antigen-binding fragment thereof.

The CARs generally include an extracellular antigen binding domain that includes, is, or is comprised within, one of the provided anti-Siglec-9 antibodies. Thus, the CARs typically include in their extracellular portions one or more Siglec-9-binding molecules, such as one or more antigen-binding fragment, domain, or portion, or one or more antibody variable regions, and/or antibody molecules, such as those described herein. In some embodiments, the CAR includes a Siglec-9-binding portion or portions of the antibody molecule, such as a heavy chain variable (VH) region and/or light chain variable (VL) region of the antibody, e.g., an scFv antibody fragment.

In some embodiments, the CAR comprising an antibody (e.g., antigen-binding fragment) provided herein, further includes a spacer, which can include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component (e.g., scFv) and the transmembrane domain. The spacer can be of a length that provides for increased responsiveness of the cell following antigen binding, as compared to in the absence of the spacer. Exemplary spacers include those having at least about 10 to 250 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 120 amino acids or less, or about 250 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687.

The antigen-recognition component generally is linked to one or more intracellular signaling components, such as signaling components that mimic activation through an antigen receptor complex, such as a TCR complex, in the case of a CAR, and/or signal via another cell surface receptor. Thus, in some embodiments, the BCMA-binding molecule (e.g., antibody or antigen-binding fragment thereof) is linked to one or more transmembrane domains such as those described herein and intracellular signaling domains comprising one or more intracellular components such as those described herein. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some embodiments, the transmembrane domain is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

The CAR generally includes an intracellular signaling domain comprising at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the Siglec-9-binding antibody is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CAR includes a chimeric molecule between CD3-zeta (CD3-0 or Fc receptor γ and CD8, CD4, CD25 or CD16.

In some embodiments, upon ligation of the CAR, the cytoplasmic domain or intracellular signaling domain of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling domain in the CAR contain(s) a cytoplasmic signaling domain, portion thereof, or sequence derived from CD3 zeta.

In some embodiments, the CAR includes a signaling domain (e.g., an intracellular signaling domain) and/or transmembrane portion of a costimulatory molecule, such as a T cell costimulatory molecule. Exemplary costimulatory molecules include CD28, 4-1BB, OX40, DAP10, and ICOS. In some aspects, the same CAR includes both the activating or stimulatory components (e.g., cytoplasmic signaling sequence) and costimulatory components.

In some aspects, the present disclosure provides methods of preparing a cell expressing a chimeric antigen receptor comprising transfecting a cell with the polynucleotides disclosed herein (e.g., anti-Siglec-9 CAR construct). In some aspects, the cell comprises a T cell, a B cell, a regulatory T cell (Treg), a tumor-infiltrating lymphocyte (TIL), a natural killer (NK) cell, a natural killer T (NKT) cell, a stem cell, an induced pluripotent stem cell, and any combination thereof.

In some aspects, the present disclosure provides a method of expanding a cell expressing a chimeric antigen receptor comprising culturing a cell comprising a polynucleotide disclosed herein or a vector disclosed herein or a polypeptide disclosed herein, under suitable conditions.

In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (e.g., CD3 zeta). In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule.

In some aspects, the CAR comprises a chimeric fusion protein comprising an antigen-binding domain, a transmembrane domain, and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

While the present application often uses CARs to illustrate the different aspects of the disclosed subject matter, it will be apparent to a skilled artisan that the relevant disclosures provided herein can equally apply to other chimeric binding proteins. As used herein, the term “chimeric binding protein” refers to proteins that are capable of binding to one or more antigens (e.g., comprising an antigen-binding moiety) and are created through the joining of two or more heterologous polynucleotides which originally coded for separate proteins or fragments of proteins or multiple fragments of the same protein connected in a non-naturally occurring orientation. Non-limiting examples of other chimeric binding proteins include a T cell receptor (TCR) (e.g., engineered TCR), chimeric antibody-T cell receptor (caTCR), chimeric signaling receptor (CSR), T cell receptor mimic (TCR mimic), and combinations thereof. Accordingly, unless indicated otherwise, the term CARs, in some aspects, can encompass other types of chimeric binding proteins known in the art, e.g., those described herein.

In some aspects, the intracellular signaling domain can comprise a primary intracellular signaling domain. Exemplary primary intracellular signaling domains include those derived from the molecules responsible for primary stimulation, or antigen dependent simulation. In some aspects, the intracellular signaling domain can comprise a costimulatory intracellular domain. Exemplary costimulatory intracellular signaling domains include those derived from molecules responsible for costimulatory signals, or antigen independent stimulation. For example, in the case of a CAR T cell (e.g., anti-Siglec-9 CAR T cells described herein), a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (ICOS), FcεRI, CD66d, CD32, DAP10, and DAP12.

### G. Therapeutic Combinations

The S9 antibodies of the invention can be used in therapeutic combinations with other therapies, such as radiation, chemotherapy, or immunotherapy. This strategy leverages the unique mechanisms of action offered by the anti-Siglec-9 antibodies presented herein—namely, their ability to modulate the immune environment—to synergize with the direct cytotoxic effects of chemotherapy and/or the targeted destruction of cancer cells by radiation. Furthermore, when used alongside other immunotherapies, anti-Siglec-9 antibodies can potentially enhance the immune system's recognition and eradication of cancer cells, particularly by overcoming immune suppression within the tumor microenvironment. For instance, combining anti-Siglec-9 therapies with PD-1/PD-L1 inhibitors could result in a more robust activation of T cells, while the addition of cytokine therapy might further stimulate the immune response against the tumor.

In some embodiments, an anti-Siglec-9 antibody or antigen-binding fragment thereof, or pharmaceutical composition, is administered to a patient as provided above, and further in combination with an additional therapeutic agent, e.g., a chemotherapeutic agent or an immune stimulating agent, such as a T cell checkpoint inhibitor. In some embodiments, the immunotherapy targets one or more target proteins selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, BTLA, CD30, TIGIT, VISTA, KIR, GAL9, TIM1, TIM3, TIM4, A2AR, LAG3, DR-5, CD2, CD3, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof. In an exemplary embodiment, the additional therapeutic agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). Suitable PD-1 antibodies also include, for example, camrelizumab (SHR-1210), tislelizumab (BGB-A317), or spartalizumab (NPVPDR001, NVS240118, PDR001). The additional therapeutic agent may also include pidilizumab (CT-011). A recombinant protein composed of the extracellular domain of PD-L2 (B7-DC) fused to the Fc portion of IgGI, called AMP-224, can also be used to antagonize the PD-1 receptor.

In another exemplary embodiment, the additional therapeutic agent is a PD-L1 antagonist, such as an antagonistic PD-L1 antibody. Suitable PD-L1 antibodies include, for example, TECENTRIQ (atezolizumab), durvalumab (MEDI4736), BMS-936559 (WO2007/005874), MSB0010718C (WO2013/79174) or rHigM12B7.

In some embodiments, the therapeutic agent is selected from the group consisting of PARP inhibitors, EGFR inhibitors, ALK inhibitors, MEK inhibitors, VEGF inhibitors, and HER2 inhibitors including trastuzumab, and any combination thereof.

In some embodiments, the therapeutic agent is selected from the group consisting of Alkylating Agents (Cyclophosphamide, Melphalan), Antimetabolites (Methotrexate, 5-Fluorouracil/5-FU), Antitumor Antibiotics (Doxorubicin, Bleomycin), Topoisomerase Inhibitors (Irinotecan, Exatecan, Deruxtecan, Etoposide/VP-16), Mitotic Inhibitors (Paclitaxel/Taxol and Docetaxel/Taxotere, Vincristine), Platinum-based Drugs (Carboplatin, Cisplatin), and any combination thereof.

### H. Diagnostic and Prognostic Applications of Anti-Siglec-9 Antibodies

Beyond therapeutic applications, the anti-Siglec-9 antibodies described herein also hold significant potential for diagnostic and prognostic purposes in oncology and immunology. Given Siglec-9's involvement in immune regulation and its expression in various diseases, these antibodies can be utilized to detect Siglec-9 expression levels in patient samples, offering insights into disease state, progression, and patient prognosis. This application extends to the use of anti-Siglec-9 antibodies in immunoassays, such as flow cytometry, ELISA, or immunohistochemistry, to quantitatively or qualitatively assess Siglec-9 presence in tissues, cells, or bodily fluids, facilitating the identification of disease biomarkers and enabling personalized medicine approaches by tailoring treatments to the patient's specific disease profile.

An anti-Siglec-9 antibody or antigen-binding fragment thereof described herein can be used to assay Siglec-9 protein levels in a subject or biological sample. In some embodiments, the subject is a human. In some embodiments, the subject is a human patient suffering from, or at risk for developing a disease, disorder, or injury of the present disclosure. Other methods include, for example, monitoring the progression of a disease, disorder or injury, by (a) assaying the expression of Siglec-9 in cells or in a tissue sample of a subject obtained at a first time point and later time point using a Siglec-9-binding molecule and (b) comparing the level of expression of Siglec-9 in the cells or in the tissue sample of the subject at the first and later time points, wherein an increase in the assayed level of Siglec-9 at the later time point compared to the first time point is indicative of the progression of disease, disorder or infection. In some embodiments, the diagnostic methods involve detecting a Siglec-9 protein in a biological sample, such as a biopsy specimen, a tissue, or a cell. A Siglec-9 agent of the present disclosure (e.g., an anti-Siglec-9 antibody described herein) is contacted with the biological sample and antigen-bound antibody is detected. For example, a biopsy specimen may be stained with an anti-Siglec-9 antibody described herein in order to detect and/or quantify disease-associated cells. Antibody detection in biological samples may occur using classical methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin. Such labels can be used to label an antibody or antigen-binding fragment thereof described herein. Alternatively, a second antibody or antigen-binding fragment thereof that recognizes an anti-Siglec-9 antibody or antigen-binding fragment thereof described herein can be labeled and used in combination with an anti-Siglec-9 antibody or antigen-binding fragment thereof to detect Siglec-9 protein levels.

Assaying for the expression level of Siglec-9 protein is intended to include qualitatively or quantitatively measuring or estimating the level of a Siglec-9 protein in a first biological sample either directly (e.g., by determining or estimating absolute protein level) or relatively (e.g., by comparing to the disease associated protein level in a second biological sample). Siglec-9 polypeptide expression level in the first biological sample can be measured or estimated and compared to a standard Siglec-9 protein level, the standard being taken from a second biological sample obtained from an individual not having the disorder or being determined by averaging levels from a population of individuals not having the disorder. As will be appreciated in the art, once the “standard” Siglec-9 polypeptide level is known, it can be used repeatedly as a standard for comparison.

As used herein, the term “biological sample” refers to any biological sample obtained from a subject, cell line, tissue, or other source of cells potentially expressing Siglec-9. Methods for obtaining tissue biopsies and body fluids from animals (e.g., humans) are well known in the art. Biological samples include peripheral mononuclear blood cells. A biological sample may also be a blood sample, in which circulating tumor cells (or “CTCs”) may express Siglec-9 and be detected.

An anti-Siglec-9 antibody or antigen-binding fragment thereof described herein can be used for prognostic, diagnostic, monitoring and screening applications, including in vitro and in vivo applications well known and standard to the skilled artisan and based on the present description. Prognostic, diagnostic, monitoring and screening assays and kits for in vitro assessment and evaluation of immune system status and/or immune response may be utilized to predict, diagnose and monitor to evaluate patient samples including those known to have or suspected of having an immune system-dysfunction or cancer. This type of prognostic and diagnostic monitoring and assessment is already in practice utilizing antibodies against the HER2 protein in breast cancer (HercepTest™, Dako) where the assay is also used to evaluate patients for antibody therapy using Herceptin®. In vivo applications include directed cell therapy and immune system modulation and radio imaging of immune responses.

Anti-Siglec-9 antibodies and antigen-binding fragments thereof described herein can carry a detectable or functional label. When fluorescence labels are used, currently available microscopy and fluorescence-activated cell sorter analysis (FACS) or a combination of both methods procedures known in the art may be utilized to identify and to quantitate the specific binding members. Anti-Siglec-9 antibodies or antigen-binding fragments thereof described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-Siglec-9 antibody can carry a radioactive label, such as the isotopes 3H, 14C, 32P, 35S, 36Cl, 51Cr, 57Co, 58Co, 59Fe, 67Cu, 90Y, 99Tc, 111In, 117Lu, 121I, 124I, 125I, 131I, 198Au, 211At, 213Bi, 225Ac and 186Re. When radioactive labels are used, currently available counting procedures known in the art may be utilized to identify and quantitate the specific binding of anti-Siglec-9 antibody or antigen-binding fragment to Siglec-9. In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-Siglec-9 antibody or antigen-binding fragment thereof under conditions that allow for the formation of a complex between the antibody or antigen-binding fragment thereof and Siglec-9. Any complexes formed between the antibody or antigen-binding fragment thereof and Siglec-9 are detected and compared in the sample and the control. In light of the specific binding of the antibodies or antigen-binding fragments thereof described herein for Siglec-9, the antibodies or antigen-binding fragments thereof can be used to specifically detect Siglec-9 expression on the surface of cells. The antibodies or antigen-binding fragments thereof described herein can also be used to purify Siglec-9 via immunoaffinity purification.

Also included herein is an assay system which may be prepared in the form of a test kit for the quantitative analysis of the extent of the presence of, for instance, Siglec-9. The system or test kit may comprise a labeled component, e.g., a labeled antibody or antigen-binding fragment, and one or more additional immunochemical reagents.

In some aspects, methods for in vitro detecting Siglec-9 in a sample, comprising contacting said sample with an antibody or antigen-binding fragment thereof, are provided herein. In some aspects, provided herein is the use of an antibody or antigen-binding fragment thereof provided herein, for in vitro detecting Siglec-9 in a sample. In one aspect, provided herein is an antibody or antigen-binding fragment thereof or pharmaceutical composition provided herein for use in the detection of Siglec-9 in a subject or a sample obtained from a subject. In one aspect, provided herein is an antibody or antigen-binding fragment thereof or pharmaceutical composition provided herein for use as a diagnostic. In one preferred embodiment, the antibody comprises a detectable label. In one preferred embodiment, Siglec-9 is human Siglec-9. In one preferred embodiment, the subject is a human.

### I. Pharmaceutical Compositions

Siglec-9 agents of the present disclosure, such as anti-Siglec-9 antibodies of the present disclosure, can be incorporated into a variety of formulations for therapeutic administration by combining the agents, such as anti-Siglec-9 antibodies, with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH-adjusting and buffering agents, toxicity-adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained-release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of an agent of the present disclosure, such as an anti-Siglec-9 antibody of the present disclosure, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the present disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

An antibody or antigen-binding fragment thereof or composition described herein can be delivered to a subject by a variety of routes, such as parenteral, subcutaneous, intravenous, intradermal, transdermal, transmucosal, intramuscular, intranasal, intratumoral, and administration to a tumor draining lymph node. In one embodiment, the antibody or antigen-binding fragment thereof or composition is administered by an intravenous route.

The amount of an antibody or antigen-binding fragment thereof or composition which will be effective in the treatment of a condition will depend on the nature of the disease. The precise dose to be employed in a composition will also depend on the route of administration, and the seriousness of the disease.

### J. Methods

The present disclosure also provide methods for using one or more of the compositions described herein (e.g., an antibody or antigen-binding fragment thereof that binds to Siglec-9 protein, a conjugate, a CAR, a cell expressing a CAR, a polynucleotide, a vector comprising the polynucleotide, or a cell transduced with the vector as described herein). While the disclosures provided below largely refer to the administration of antibodies (e.g., anti-Siglec-9 antibodies), it will be apparent to those skilled in the art that the described methods can be achieved by administering to the subject any of the other compositions described herein.

Further aspects of the present disclosure provide methods of inhibiting one or more Siglec-9 activities, including with limitation, inhibiting a Siglec-9 protein of the present disclosure; counteracting one or more phosphorylation of Tyr-433 and Tyr-456 by a Src family tyrosine kinase, such as Syk, LCK, FYM, and/or ZAP70; recruitment of and binding to the tyrosine-specific protein phosphatases SHP1 and SHP2; recruitment of and binding to PLC-gammal, which acts as a guanine nucleotide exchange factor for Dynamini-1; recruitment of and binding to SH2-domain containing protein (e.g., Crkl); recruitment of and binding to the spleen tyrosine kinase Syk; recruitment of and binding to SH3-SH2-SH3 growth factor receptor-bound protein 2 (Grb2); recruitment of and binding to multiple SH2-containing proteins; inhibiting expression of one or more pro-inflammatory cytokines, optionally wherein the one or more anti-inflammatory cytokines are selected from FN-α4, IFN-beta, IL-1β, IL-1alpha, TNF-α, IL-6, IL-8, CRP, IL-20 family members, LIF, IFN-γ, OSM, CNTF, GM-CSF, IL-11, IL-12, IL-17, IL-18, IL-33, MCP-1, and MIP-1-beta; inhibiting expression of one or more pro-inflammatory cytokines in one or more cells selected from macrophages, neutrophils, NK cells, dendritic cells, bone marrow-derived dendritic cells, monocytes, osteoclasts, T cells, T helper cells, cytotoxic T cells, granulocytes, and microglial cells; inhibiting expression of one or more anti-inflammatory cytokines, optionally wherein the one or more anti-inflammatory cytokines are selected from IL-4, IL-10, IL-13, IL-35, IL-16, TGF-beta, IL-1Ra, G-CSF, and soluble receptors for TNF, IFN-beta1a, IFN-beta1b, or IL-6; inhibiting expression of one or more anti-inflammatory cytokines in one or more cells selected from macrophages, neutrophils, NK cells, dendritic cells, bone marrow-derived dendritic cells, monocytes, osteoclasts, T cells, T helper cells, cytotoxic T cells, granulocytes, and microglial cells; inhibiting expression of one or more proteins selected from C1qa, C1qB, C1qC, C1s, C1R, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, and PYCARD; activation of extracellular signal-regulated kinase (ERK) phosphorylation; inhibiting tyrosine phosphorylation on one or more cellular proteins, optionally, wherein the one or more cellular proteins comprise ZAP-70 and the tyrosine phosphorylation occurs on Tyr-319 of ZAP-70; inhibiting expression of CC chemokine receptor 7 (CCR7); activation of microglial cell chemotaxis toward CCL19-expressing and CCL21-expressing cells; inhibiting T cell proliferation induced by one or more cells selected from dendritic cells, bone marrow-derived dendritic cells, monocytes, microglia, M1 microglia, activated M1 microglia, M2 microglia, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, and M2 NK cells; inhibiting osteoclast production, inhibiting rate of osteoclastogenesis, or both; inhibiting survival of one or more cells selected from dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia; inhibiting proliferation of one or more cells selected from dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia; inhibiting migration of one or more cells selected from dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia; inhibiting one or more functions of one or more cells selected from dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia; inhibiting maturation of one or more cells selected from dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia; activation of one or more types of clearance selected from apoptotic neuron clearance, nerve tissue debris clearance, dysfunctional synapse clearance, non-nerve tissue debris clearance, bacteria clearance, other foreign body clearance, disease-causing protein clearance, disease-causing peptide clearance, and tumor cell clearance; optionally wherein the disease-causing protein is selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein Al, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides and the tumor cell is from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer; activation of phagocytosis of one or more of apoptotic neurons, nerve tissue debris, dysfunctional synapses non-nerve tissue debris, bacteria, other foreign bodies, disease-causing proteins, disease-causing peptides, disease-causing nucleic acids, or tumor cells; optionally wherein the disease-causing nucleic acids are antisense GGCCCC (G2C4) repeat-expansion RNA, the disease-causing proteins are selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein Al, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the tumor cells are from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, or thyroid cancer; inhibiting binding to Siglec-9 ligand on tumor cells; inhibiting binding to Siglec-9 ligand on cells selected from neutrophils, dendritic cells, bone marrow-derived dendritic cells, monocytes, microglia, macrophages, and NK cells; activation of tumor cell killing by one or more of microglia, macrophages, neutrophils, NK cells, dendritic cells, bone marrow-derived dendritic cells, neutrophils, T cells, T helper cells, or cytotoxic T cells; activating anti-tumor cell proliferation activity of one or more of microglia, macrophages, neutrophils, NK cells, dendritic cells, bone marrow-derived dendritic cells, neutrophils, T cells, T helper cells, or cytotoxic T cells; activation of anti-tumor cell metastasis activity of one or more of microglia, macrophages, neutrophils, NK cells, dendritic cells, bone marrow-derived dendritic cells, neutrophils, T cells, T helper cells, or cytotoxic T cells; inhibiting of one or more ITAM motif containing receptors, optionally wherein the one or more ITAM motif containing receptors are selected from TREM1, TREM2, Sirp beta, FcgR, DAP10, and DAP12; inhibiting of signaling by one or more pattern recognition receptors (PRRs), optionally wherein the one or more PRRs are selected from receptors that identify pathogen-associated molecular patterns (PAMPs), receptors that identify damage-associated molecular patterns (DAMPs), and any combination thereof, inhibiting of signaling by one or more Toll-like receptors; inhibiting of the JAK-STAT signaling pathway; inhibiting of nuclear factor kappa-light-chain-enhancer of activated B cells (NFκB); phosphorylation of an ITAM motif containing receptor; inhibiting expression of one or more inflammatory receptors, proteins of the complement cascade, and/or receptors, optionally wherein the one or more inflammatory receptors, proteins of the complement cascade, and/or receptors comprise CD86, C1qa, C1qB, C1qC, C1s, C1R, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, and/or PYCARD, and the one or more inflammatory receptors, proteins of the complement cascade, and/or receptors are expressed on one or more of microglia, macrophages, neutrophils, NK cells, dendritic cells, bone marrow-derived dendritic cells, neutrophils, T cells, T helper cells, or cytotoxic T cells; inhibiting expression of one or more Siglec-9-dependent genes; normalization of disrupted Siglec-9-dependent gene expression; inhibiting expression of one or more ITAM-dependent genes, optionally wherein the one more ITAM-dependent genes are activated by nuclear factor of activated T cells (NFAT) transcription factors; rescuing functionality of one or more of immunosuppressor dendritic cells, immunosuppressor macrophages, immunosuppressor neutrophils, immunosuppressor NK cells, myeloid-derived suppressor cells, tumor-associated macrophages, tumor-associated neutrophils, tumor-associated NK cells, and regulatory T cells; reducing infiltration of one or more of immunosuppressor dendritic cells, immunosuppressor macrophages, immunosuppressor neutrophils, immunosuppressor NK cells, myeloid-derived suppressor cells, tumor-associated macrophages, tumor-associated neutrophils, tumor-associated NK cells, and regulatory T cells into tumors; increasing the number of tumor-promoting myeloid/granulocytic immune-suppressive cells in a tumor, in peripheral blood, or other lymphoid organ; enhancing tumor-promoting activity of myeloid-derived suppressor cells; increasing expression of tumor-promoting cytokines in a tumor or in peripheral blood, optionally wherein the tumor-promoting cytokines are TGF-beta or IL-10; increasing tumor infiltration of tumor-promoting FoxP3+ regulatory T lymphocytes; enhancing tumor-promoting activity of myeloid-derived suppressor cells (MDSC); decreasing activation of tumor-specific T lymphocytes with tumor killing potential; decreasing infiltration of tumor-specific NK cells with tumor killing potential; decreasing the tumor killing potential of NK cells; decreasing infiltration of tumor-specific B lymphocytes with potential to enhance immune response; decreasing infiltration of tumor-specific T lymphocytes with tumor killing potential; increasing tumor volume; increasing tumor growth rate; increasing metastasis; increasing rate of tumor recurrence; decreasing efficacy of one or more immune-therapies that modulate anti-tumor T cell responses, optionally wherein the one or more immune-therapies are immune-therapies that target one or more target proteins selected from PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIM1, TIM3, TIM4, A2AR, LAG3, DR-5, CD2, CD3, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, phosphatidylserine, and any combination thereof, or of one or more cancer vaccines; inhibition of PLCγ/PKC/calcium mobilization; and/or inhibition of PI3K/Akt, Ras/MAPK signaling in an individual in need thereof, by administering to the individual a therapeutically effective amount of an anti-Siglec-9 composition of the present disclosure, such as an anti-Siglec-9 antibody of the present disclosure, to inhibit one or more of the Siglec-9 activities in the subject.

In some aspects, the methods of blocking the binding of Siglec-9 protein to sialic acid in cells comprise contacting the cells with the anti-Siglec-9 compositions of the present disclosure. In some aspects, the cells can be selected from the group consisting of dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia. In some aspects, the cells are tumor cells.

In some aspects, the methods of inhibiting one or more activities of Siglec-9 protein in cells comprise contacting the cells with the anti-Siglec-9 compositions of the present disclosure. In some aspects, the cells can be selected from the group consisting of dendritic cells, bone marrow-derived dendritic cells, macrophages, neutrophils, NK cells, M1 macrophages, M1 neutrophils, M1 NK cells, activated M1 macrophages, activated M1 neutrophils, activated M1 NK cells, M2 macrophages, M2 neutrophils, M2 NK cells, monocytes, osteoclasts, T cells, T helper cells, cytotoxic T cells, granulocytes, neutrophils, microglia, M1 microglia, activated M1 microglia, and M2 microglia. In some aspects, the cells are tumor cells.

In some aspects, the present disclosure provides methods of treating or preventing a Siglec-9-associated disorder. In some aspects, the disorder is selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express Siglec-9, thyroid cancer, infections, CNS herpes, parasitic infections, Trypanosoma cruzi infection, Pseudomonas aeruginosa infection, Leishmania donovani infection, group B Streptococcus infection, Campylobacter jejuni infection, Neisseria meningiditis infection, type I HIV, SARS-CoV-2 infection, and Haemophilus influenza, by administering to an subject in need thereof a therapeutically effective amount of an agent of the present disclosure that inhibits interaction between Siglec-9 and one or more Siglec-9 ligands. In some aspects, the agent is an anti-Siglec-9 antibody of the present disclosure.

In some embodiments, the present disclosure provides methods of preventing, reducing risk, or treating cancer, by administering to an individual in need thereof, a therapeutically effective amount of an agent of the present disclosure that inhibits interaction between Siglec-9 and one or more Siglec-9 ligands. In some aspects, the agent is an anti-Siglec-9 antibody of the present disclosure.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer ofunknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In some aspects, the cancer or tumor is stage II or stage III, such that, e.g., the cancer or tumor is larger than in stage 0 or stage I, and it has grown into neighboring tissues but it has not metastasized, except potentially to the lymph nodes. In some aspects, the cancer or tumor is stage IV, such that, e.g., the cancer or tumor has metastasized. Stage IV can also be referred to as advanced or metastatic cancer.

In some aspects, the tumor is a solid tumor. A “solid tumor” includes, but is not limited to, sarcoma, melanoma, carcinoma, or other solid tumor cancer. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

In further embodiments, an anti-Siglec-9 antibody or antigen-binding fragment thereof, or pharmaceutical composition, is administered to a patient as provided above, and further in combination with an additional therapeutic agent, e.g., a chemotherapeutic agent or immunotherapy. In some embodiments, the immunotherapy targets one or more target proteins selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, BTLA, CD30, TIGIT, VISTA, KIR, GAL9, TIM1, TIM3, TIM4, A2AR, LAG3, DR-5, CD2, CD3, CD5, TREM1, TREM2, CD39, CD73, CSF-1 receptor, Siglec-7, Siglec-10, Siglec-15, SIRPα, CD47, NKG2A, TGFBR, IDO1, STING, phosphatidylserine, and any combination thereof.

Usually, the patient is a human but non-human mammals including transgenic mammals can also be treated.

An antibody or antigen-binding fragment thereof or composition described herein can be delivered to a subject by a variety of routes, such as parenteral, subcutaneous, intravenous, intradermal, transdermal, intranasal, intratumoral, and administration to a tumor draining lymph node. In one embodiment, the antibody or antigen-binding fragment thereof or composition is administered by an intravenous route.

The amount of an antibody or antigen-binding fragment thereof or composition which will be effective in the treatment of a condition will depend on the nature of the disease. The precise dose to be employed in a composition will also depend on the route of administration, and the seriousness of the disease.

In some embodiments, the methods of the present disclosure may further involve the co-administration of Siglec-9 agents, such as anti-Siglec-9 antibodies or bispecific anti-Siglec-9 antibodies, with antibodies that bind to another antigen, such as inhibitory cytokine.

### K. Kits

Provided herein are kits comprising one or more antibodies or antigen-binding fragments thereof described herein or conjugates thereof. In a specific embodiment, provided herein is a pharmaceutical pack or kit comprising one or more containers filled with one or more of the ingredients of the pharmaceutical compositions described herein, such as one or more antibodies or antigen-binding fragments thereof provided herein. Optionally associated with such container(s) can be a notice in the form prescribed by a governmental agency regulating the manufacture, use, or sale of pharmaceuticals or biological products, which notice reflects approval by the agency of manufacture, use or sale for human administration.

Also provided herein are kits that can be used in diagnostic methods. In one embodiment, a kit comprises an antibody or antigen-binding fragment thereof described herein, preferably a purified antibody or antigen-binding fragment thereof, in one or more containers. In a specific embodiment, kits described herein contain a substantially isolated Siglec-9 antigen (e.g., human Siglec-9) that can be used as a control. In another specific embodiment, the kits described herein further comprise a control antibody or antigen-binding fragment thereof which does not react with a Siglec-9 antigen. In another specific embodiment, kits described herein contain one or more elements for detecting the binding of an antibody or antigen-binding fragment thereof to a Siglec-9 antigen (e.g., the antibody or antigen-binding fragment thereof can be conjugated to a detectable substrate such as a fluorescent compound, an enzymatic substrate, a radioactive compound or a luminescent compound, or a second antibody or antigen-binding fragment thereof which recognizes the first antibody or antigen-binding fragment thereof can be conjugated to a detectable substrate). In specific embodiments, a kit provided herein can include a recombinantly produced or chemically synthesized Siglec-9 antigen. The Siglec-9 antigen provided in the kit can also be attached to a solid support. In a more specific embodiment, the detecting means of the above described kit includes a solid support to which a Siglec-9 antigen is attached. Such a kit can also include a non-attached reporter-labeled anti-human antibody or antigen-binding fragment thereof or anti-mouse/rat antibody or antigen-binding fragment thereof. In this embodiment, binding of the antibody or antigen-binding fragment thereof to the Siglec-9 antigen can be detected by binding of the said reporter-labeled antibody or antigen-binding fragment thereof

### EXAMPLES

The examples presented below are provided for the purpose of illustration only and the embodiments described herein should in no way be construed as being limited to these examples. Rather, the embodiments should be construed to encompass any and all variations which become evident as a result of the teaching provided herein.

### Example 1: Development of Anti-Siglec-9 Antibodies

To develop monoclonal antibodies that block the binding of Siglec-9 to sialic acid, a mouse immunization campaign with human recombinant Siglec-9 was conducted. It should be noted that the homology between human Siglec-9 and its mouse orthologue (Siglec-E) is low, so human Siglec-9 is a good immunogen in the mouse species

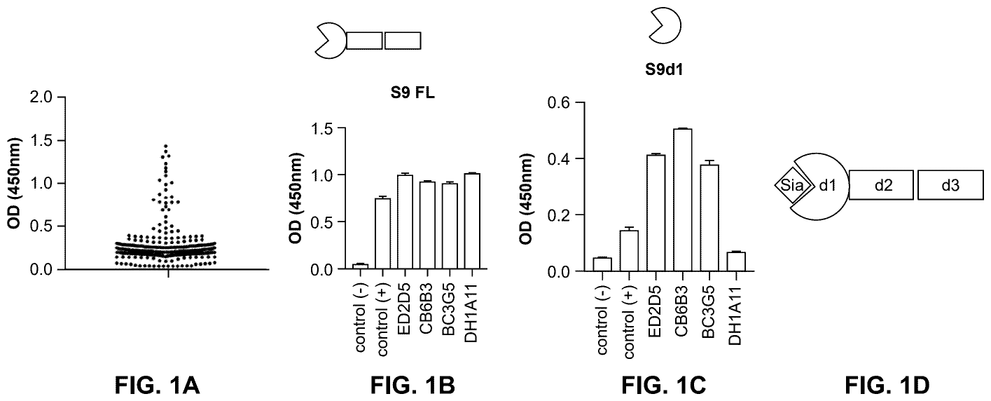

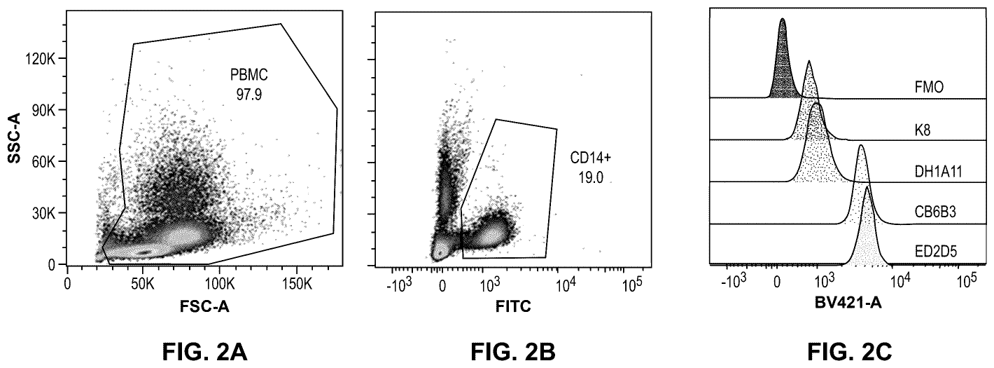

Initially, a screening was conducted that consisted of performing an ELISA against human recombinant Siglec-9, selecting the positive clones that progressed, performing another ELISA with a truncated version of Siglec-9 that only contains the V domain, which is the domain that binds to sialic acid (FIG. 1). Clones that bind to the V domain were selected (clones ED2D5, CB6B3, and BC3G5) to later evaluate their binding capacity to primary cells (CD14+) that express Siglec-9 on their surface (FIG. 2).

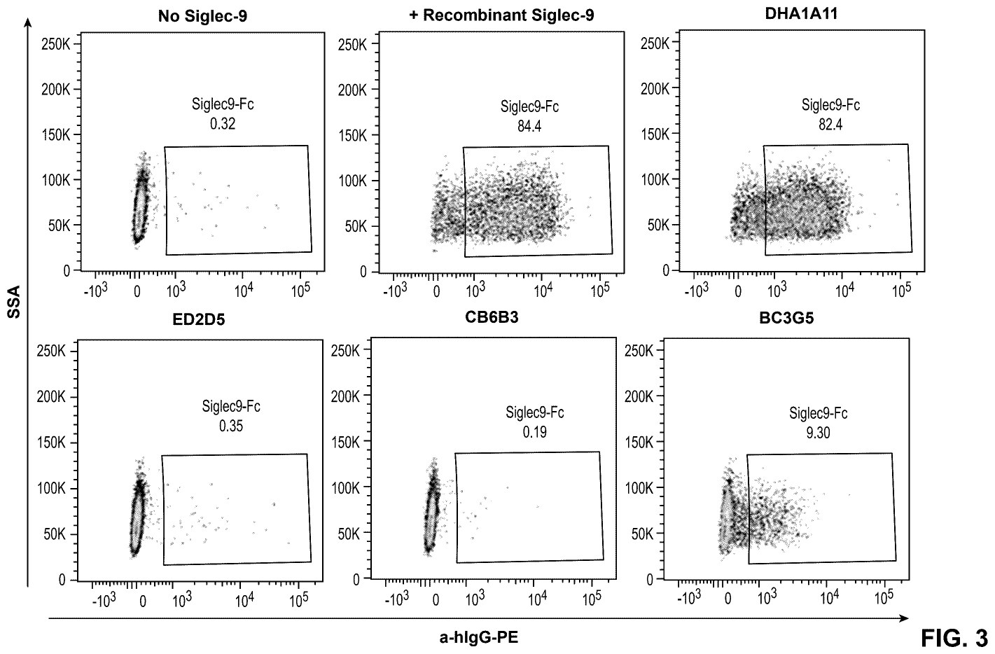

Positive clones were subjected to a selection based on their capacity to block the binding of a recombinant human Siglec-9-Fc fusion protein to Ramos lymphoma cells, which express sialic acid on their surface. As a result of this experiment, clones ED2D5 and CB6B3 were prioritized for their comparison with another existing antibody (clone mAbA) and subsequent evaluation of their affinity (FIG. 3).

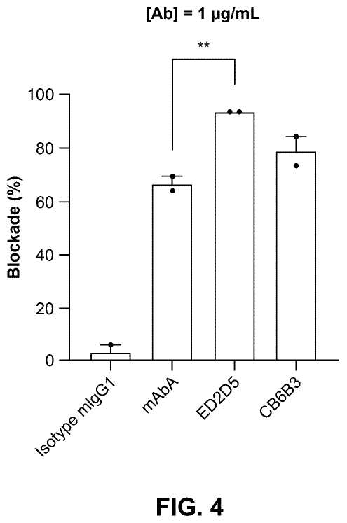

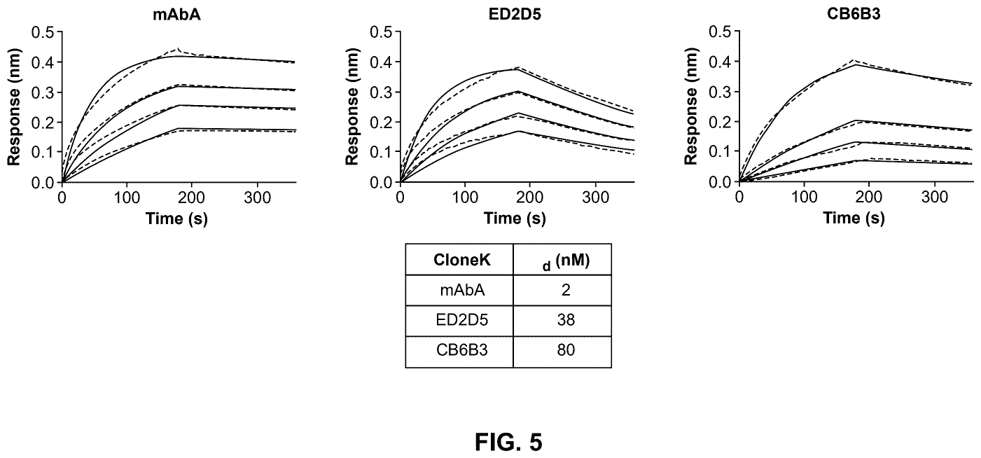

To proceed with their characterization and compare their blocking capacity against an existing clone (clone mAbA) previously described in a U.S. Pat. No. 11,078,274 by Innate pharma, a comparative blocking assay was carried out (FIG. 4) and their affinity was quantified through a bilayer interferometry assay (FIG. 5).

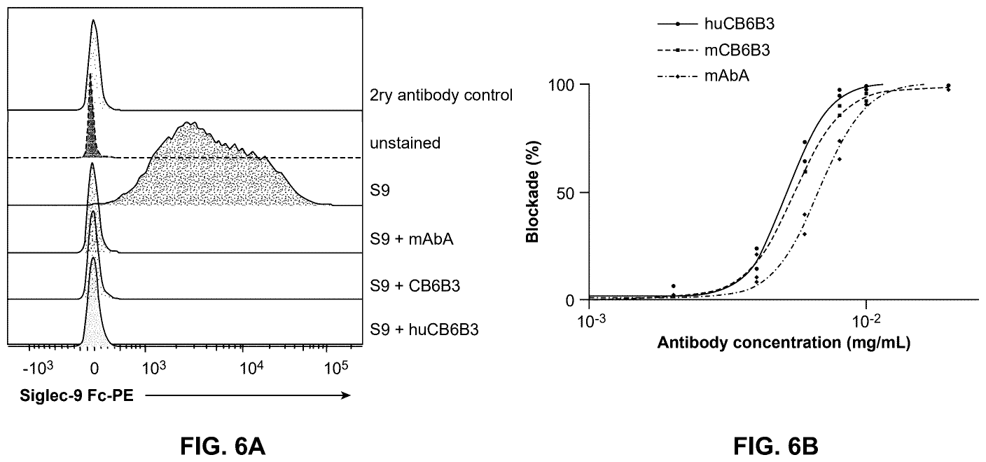

Clone CB6B3 was selected for humanization by CDR grafting. The humanized antibody (termed huCB6B3) showed comparable affinity to the parental mouse antibody, assessed by ELISA against recombinant Siglec-9. The blocking capacity of clone huCB6B3 was assessed in a cell-based assay, in which recombinant Siglec-9 binds to the surface of Ramos lymphoma cells. Each antibody was assessed for its capacity to block this interaction (FIG. 6A), showing that parental mouse CB6B3 and huCB6B3 have superior blocking capacity than clone mAbA (Innate pharma) (FIG. 6B).

This Example demonstrates that anti-Siglec-9 monoclonal antibodies (clones ED2D5 and CB6B3) with capacity of blocking the sialic acid binding domain of Siglec-9 have been generated. Clone CB6B3 was humanized, and showed a superior capacity of blocking the binding of Siglec-9 to its natural ligands than an existing mAb (clone mAbA, Innate pharma).

### Example 2: Materials and Methods

### 2.1 Recombinant Siglec-9 Protein Production and Purification

The DNA sequence encoding protein Siglec-9 (residues 18-344) domains 1 to 3 was codon optimized for expression in human HEK293F cells (Thermo Fisher Scientific). DNA synthesis and cloning was carried out by Genscript. In order to improve its affinity purification, a 6-histidine tail (6x His-tag) was added at the C-terminus.

HEK293F cells were transiently transfected with the ectoSiglec-9-pHL-see construct. Cells were divided into 200 mL culture flasks at 0.9×106 cells per mL in Freestyle Medium (Gibco). DNA (50 μg) was incubated with FectoPRO transfection reagent (Polyplus Transfections) in a ratio of 1:1 at room temperature for 10 min, and added to the cells.

After 7 days of incubation at 37° C., 180 rpm, 8% CO₂ in a Minitron Pro shaker (Infors HT), the cultures were collected by centrifugation at 4000×g for 30 minutes and then supernatants were collected and filtered with a 0.45 μm Filter (Corning). These supernatants were run through a HisTrap Ni-NTA column (GE Healthcare) at 4 mL min-1. The washing step consisted of 10 column volumes of 20 mM Tris pH 8, 150 mM NaCl and 4% elution buffer (20 mM Tris pH 8, 150 mM NaCl, 500 mM imidazole). Finally, a 100% elution buffer gradient was established. The fractions containing the eluted recombinant Siglec-9 protein were collected.

The Siglec-9 protein had the following amino acid sequence:

(SEQ ID NO: 2)
QTSKLLTMQSSVTVQEGLCVHVPCSFSYPSHGWIYPGPVVHGYWFREGAN
TDQDAPVATNNPARAVWEETRDRFHLLGDPHTKNCTLSIRDARRSDAGRY
FFRMEKGSIKWNYKHHRLSVNVTALTHRPNILIPGTLESGCPQNLTCSVP
WACEQGTPPMISWIGTSVSPLDPSTTRSSVLTLIPQPQDHGTSLTCQVTF
PGASVTTNKTVHLNVSYPPQNLTMTVFQGDGTVSTVLGNGSSLSLPEGQS
LRLVCAVDAVDSNPPARLSLSWRGLTLCPSQPSNPGVLELPWVHLRDAAE
FTCRAQNPLGSQQVYLNVSLQSKATSG.

Fractions containing recombinant Siglec-9 protein were concentrated and processed in a Superdex 75 Increase 10/300 gel filtration column (GE Healthcare) at 0.5 mL min-1 in 20 mM Tris pH 8, 150 mM NaCl. The purity of the samples was evaluated by polyacrylamide gel electrophoresis (SDS-PAGE).

2.2 Immunization of Balb/c Mice with Recombinant Siglec-9

The immunization of two BALB/c female mice was carried out following Euromabnet recommendations. Mice received three injections of 100 ug of the purified ectodomain of recombinant Siglec-9 (SEQ ID NO: 2). A first subcutaneous (s.c.) injection comprising an emulsion of the antigen with complete Freund adjuvant was administered. After 10 days, a second s.c. injection with the antigen and incomplete Freund adjuvant was administered. 10 days after the second immunization, a s.c. injection with recombinant Siglec-9 diluted in PBS was administered. After 40 days of the initial injection, a final boost was administrated with 200 □g of recombinant Siglec-9 in PBS was administered intraperitoneally.

### 2.3 Generation of Anti-Siglec-9 Monoclonal Antibodies by Hybridoma Technology

Splenocytes were isolated from immunized mice, washed and resuspended in RPMI 1640 in a 50 mL falcon tube with myeloma cells (SP2/0-Ag14) in a 1:2 ratio. The mixture was centrifuged at 210 g during 10 minutes to obtain the pellet. The fusion take place in the presence of polyethylene glycol (PEG1500) at 37° C. Hybridomas were grown in HAT Medium and seeded in a 96 well plate. After 14 days at a 37° C. and 5% CO₂, supernatants were collected and evaluated for the presence of anti-Siglec-9 antibodies by ELISA. Positive hybridomas were cultured in a 24 well plate in HT medium. At this point, a limiting dilution was carried out to isolate single hybridoma clones. Hybridomas producing anti-Siglec-9 monoclonal antibodies were selected based on ELISA.

2.4 Hybridoma Screening by ELISA Coated with Siglec-9

96-well plates were coated with recombinant Siglec-9 (100 μL) at 2 g/mL in carbonate/bicarbonate buffer pH 9.6 overnight at 4° C., and washed with 250 μL of PBST (PBS, 0.1% Tween-20). Plates were blocked with blocking buffer (3% BSA in PBS) for 1 hour at room temperature, washed, and 100 μl of hybridoma supernatants were added for 2 hours at RT. The plates were washed and incubated at room temperature with an anti-mouse IgG HRP detection antibody (1:5000, 1 hour, Anti-mouse IgG-HRP), followed by three washes of 250 μl of PBST, and two more with 400 μl of PBS, before the addition of 100 μl of TMB (Thermo Scientific) for 2-6 minutes. The reaction was stopped with 50 μl of Stop Solution (Thermo Scientific). The optical density (OD) was measured at 450 nm in a multimode plate reader (Victor Nivo, PerkimElmer). Antibdies binding to Siglec-9 were selected for downstream characterization.

2.5 Measurement of Binding Capacity of Monoclonal Antibodies (mABs) to Cells Expressing Siglec-9

The capacity of the binding of anti-Siglec-9 mAbs to Siglec-positive cells was evaluated using the K562 cell line expressing full length Siglec-9. The assay was carried out by incubating K562 cells with anti-Siglec-9 mAbs for 30 minutes at 4° C. in the dark. Cells were then washed and incubated with an anti-mouse IgG1-BV421 secondary antibody (1:200) for 30 minutes at 4° C. in the dark. After a final wash, cells were resuspended in 200 μL of 2% BSA in PBS with DAPI (1:10000). Cells were acquired in a FACSymphony flow cytometer and analyzed using FlowJo (BD Biosciences).

### 2.6 Humanization of CB6B3 mAb

Hybridoma sequencing was carried out by Absolute Antibody Ltd. (Wilton Centre Redcar, Cleveland TS10 4RF United Kingdom) The VH and VL domains of antibody CB6B3 were humanized by CDR grafting. In a first step, the closest murine germlines for the VH and VL segments were retrieved to identify mutations introduced during affinity maturation. In total, 12 mutations were identified in the VH segment and 4 in the VL segment. Then, a search was performed to identify suitable human germline genes for the VH and VL segments. Two different human VH segments and two different VL segments were chosen for CDR grafting (all with a positive Z-score for humanness), followed by further analysis and modifications to retain CDR structures. For VH1, two different variants (VH1 and VH1b) were generated with VH1b differing by a shorter and more restricted CDRH1.

### 2.7 Antibody Production

In total, seven different antibodies were produced. One chimeric IgG1 (cIgG) and six humanized variants. The corresponding heavy chains (HC) and light chains (LC) were cloned into expression vector pSecTagAL1 (a modified version of pSecTagA) and produced in transiently transfected suspension HEK293-6E cells in a total volume of 100 mL. After 5 days, supernatants were collected and antibodies were purified by protein A beads, eluted with 100 mM glycine pH 3.5. Yields ranged between 0.9 and 16.0 mg/L supernatant. Increased productivity was observed for antibodies containing the second variant of VL (VL2).

### 2.8 Quality Control (SDS-PAGE)

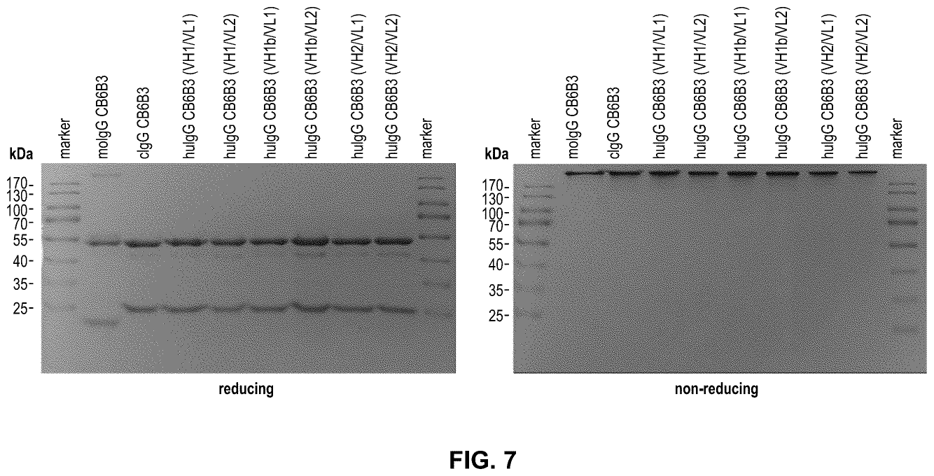

All antibodies were analyzed by SDS-PAGE under reducing and non-reducing conditions (FIG. 7). Under reducing conditions, one major band at approximately 55 kDa and one slighter band at in the range between 26 and 23 kDa were observed corresponding to the heavy chain and the light chain, respectively. For the mouse antibody moIgG CB6B3, one further faint band above 170 kDa was detected. For the other antibodies (cIgG and huIgG variants), a faint band at approximately 50 kDa (below the HC band) was detected, which might be due to differences in N-glycosylation of the CH2 domain. Under non-reducing conditions, one major band at approximately 200 kDa was observed representing the intact dimeric antibody containing two heavy and two light chains. In summary, SDS-PAGE confirmed a high purity and correct assembly of the different antibodies.

### 2.9 Quality Control (Size Exclusion Chromatography)

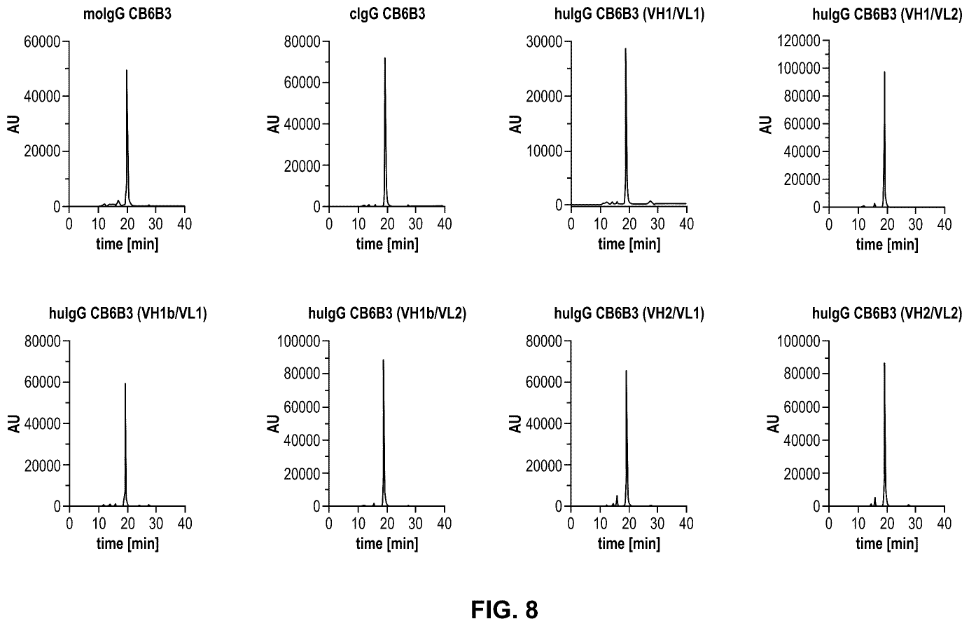

The different antibodies were further analyzed by HPLC size-exclusion chromatography (SEC) using a TSKgel SuperSW mAb HR column (FIG. 8). SEC analysis of all antibodies revealed one major peak at approximately 200 kDa representing the correctly assembled antibody under native conditions. In addition, minor peaks of high molecular weight were detected for the different antibodies. The integrity of the different intact antibodies was 87.1% for moIgG CB6B3, 96.4% for cIgG CB6B3, 88.5% for huIgG CB6B3 (VH1/VL1), 96.7% for huIgG CB6B3 (VH1/VL2), 94.9% for huIgG CB6B3 (VH1b/VL1), 97.6% for huIgG CB6B3 (VH1b/VL2), 88.7% for huIgG CB6B3 (VH2/VL1) and 91.1% for huIgG CB6B3 (VH2/VL2), respectively. Thus, cIgG CB6Bb, huIgG CB6B3 (VH1/VL2), huIgG CB6B3 (VH1b/VL1) and huIgG (VH1b/VL2) exhibit >95% purity under native conditions.

### 2.10 Thermal Stability

Thermal stability of selected antibodies (moIgG CB6B3, cIgG CB6B3, and huIgG CB6B3 (VH1b/VL2)) was analyzed by dynamic light scattering (DLS) using a Zetasizer Nano ZS. For this experiment, 100 μg antibody in 1 mL PBS were sterile filtered into a glass cuvette before measuring the particle size in a temperature range of 30 to 85° C. in 1° C. intervals. Measurements were performed twice at each temperature after equilibration of the sample for 2 minutes. The aggregation point was defined as the temperature showing a clear increase in the detected mean count rate. For the murine and chimeric IgG molecules similar aggregation points were determined with 69° C. for moIgG CB6B3 and 70° C. for cIgG CB6B3. The humanised antibody huIgG CB6B3 (VH1b/VL2) showed a slightly reduced thermal stability at 64° C.

### 2.11 Antigen Binding

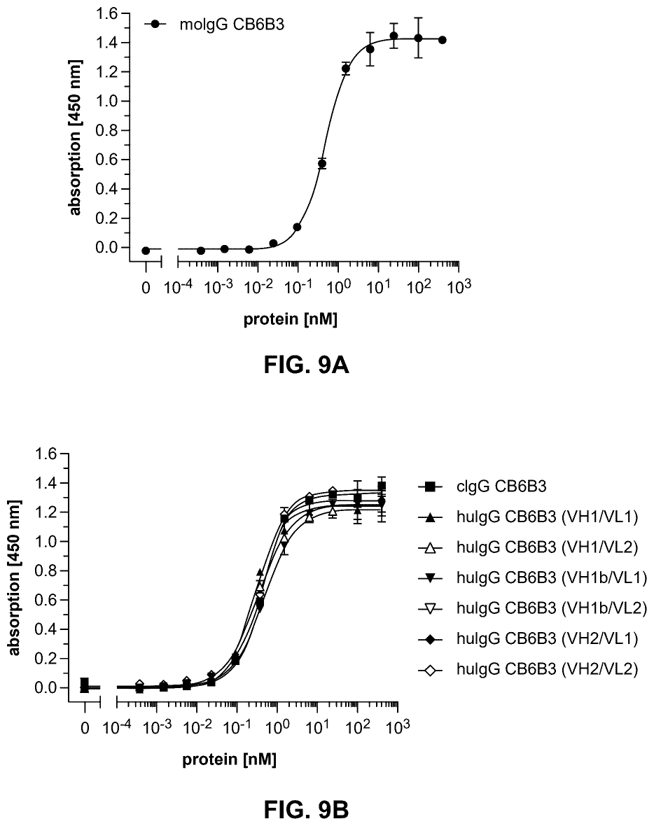

The binding of the different IgG CB6B3 antibodies was analyzed via ELISA using human Siglec-9 protein as immobilized antigen (2 μg/mL). The different antibodies were titrated from 0.4 pM to 400 nM (FIG. 9). Human Siglec-9 protein was diluted in PBS pH 7.4 and coated overnight at 4° C. Remaining binding sites were blocked with PBS, 2% skimmed milk power (MPBS) for 2 hours. Bound murine IgG CB6B3 antibody was detected with an HRP-conjugated anti-mouse Fc secondary antibody (Sigma Aldrich A554; 1:5,000 dilution). All antibodies were diluted in MPBS. Incubation times were 1 hour each. TMB/H2O2 was used as substrate. In general, all IgG molecules bound to the immobilized antigen in a concentration-dependent manner with similar EC50 values in the range between 271 pM and 500 pM. Thus, the humanised antibodies retained antigen specificity and bound with similar potency to purified antigen in ELISA.

### Example 3: In Vitro Functional Assays for the Evaluation of the Activity of Anti-Siglec-9 Monoclonal Antibodies

A co-culture assay comprising three different cell types (macrophages, tumor cells and CAR-T cells) was developed to evaluate the activity of anti-Siglec-9 monoclonal antibodies. Primary human cells were obtained from buffy coats of healthy donors (Biobanco Vasco, BIOEF) after ethical approval. Briefly, peripheral blood mononuclear cells (PBMCs) were separated by gradient differentiation using Ficoll-Histopaque (17-1440-03, Fisher scientific). CD3+ T cells were purified by negative selection using EasySep™ Human T Cell Enrichment Kit (Stemcell) following manufacturer's instructions, and CD14+ Monocytes were purified by positive selection (Stemcell). Purity was confirmed by flow cytometry (>95%). T cells were then activated with anti-CD3/CD28 Dynabeads (11131D, Thermo Fisher) in CST OpTimizer medium (A1048501, Gibco) supplemented with IL-2 at 100 IU/mL (130-097-743, Miltenyi Biotec).

CD14+ monocytes isolated from PBMCs were differentiated into macrophages using M-CSF (50 ng/mL) for 4 days. Macrophages were treated with a cytokine cocktail (IL-4, IL-6, IL-13; 20 ng/mL) and co-cultured with MDA-MB-231-hCD19t cells, a breast cancer cell line engineered to overexpress a model target antigen (human CD19). Anti-Siglec-9 monoclonal antibodies were added to the co-culture. 72 hours later, anti-CD19 CAR-T cells were added in a ratio of 1:8:8 (CAR-T:Tumor:macrophages) and their cytotoxicity was measured by luminescence assay at 72 hours in a multimode plate reader (Victor Nivo, PerkimElmer).

### Example 4: Evaluation of the Blocking Activity of Anti-Siglec-9 Antibodies

In order to evaluate the blockade capacity of the generated anti-Siglec-9 monoclonal antibodies, an assay to measure the binding of recombinant Siglec-9-Fc to Ramos lymphoma cells in the presence or absence of anti-Siglec-9 antibodies was carried out.

The assay consisted of incubating 0.5 μg of recombinant Siglec-9-Fc with anti-Siglec-9 mAbs for 30 minutes at 4° C. in the dark. 2×10⁵ Ramos lymphoma cells were added to each well for another 30 minutes at 4° C. Then, samples were washed with 2% of bovine serum albumin (BSA) in PBS and incubated with an anti-human IgG-APC secondary antibody (1:200) for 30 minutes at 4° C. in the dark. After a final wash, cells were resuspended in 200 μL of 2% BSA in PBS with DAPI (1:10000). Cells were acquired in a FACSymphony flow cytometer and analyzed using FlowJo (BD Biosciences).

For dose-response blocking assays, Ramos lymphoma cells (0.2×10⁶) were incubated with anti-Siglec-9 antibodies (clones huCB6B3, mCB6B3 and mAbA) at concentrations of 20, 10, 8, 6, 4, 2, 1, 0.3, 0 μg/mL (serial dilutions) in 2% BSA in PBS for 30 minutes at 4° C. After washing, cells were incubated with recombinant Siglec-9-Fc at 8 μg/mL for 30 minutes at 4° C. Cells were then washed and incubated with anti-human IgG Fc PE (12-4998-82, Thermo Fisher, 1:200) and incubated for 30 minutes at 4° C. in the dark. Cells were washed and resuspended in 200 μL 2% BSA in PBS with DAPI (1:20,000) before acquisition on a FACSymphony.

### Example 5: Epitope Mapping of Anti-Siglec-9 Antibodies

Epitope mapping was conducted to identify the binding regions of Siglec-9 for each generated anti-Siglec-9 monoclonal antibody. To reconstruct epitopes of the target molecule, a library of peptide-based mimics was synthesized using Fmoc-based solid-phase peptide synthesis. The process begins with grafting an amino-functionalized polypropylene support with a proprietary hydrophilic polymer formulation, followed by reaction with butyloxycarbonyl-t-hexamethylenediamine (BocHMDA) using dicyclohexylcarbodiimide (DCC) and N-hydroxybenzotriazole (HOBt). The Boc-groups are then cleaved using trifluoroacetic acid (TFA). Standard Fmoc-peptide synthesis was used to synthesize peptides on the amino-functionalized solid support.

Biosynth's proprietary Chemically Linked Peptides on Scaffolds (CLIPS) technology was employed for constrained peptides. This technology enables the structure of peptides into various configurations, including single loops, double loops, triple loops, sheet-like folds, helix-like folds, or combinations thereof. The CLIPS scaffold is coupled to cysteine residues, with the side-chains of multiple cysteines in the peptides being linked to a CLIPS scaffold containing two or three reactive groups. A 0.5 mM solution of the P2 CLIPS (2,6-bis(bromomethyl)pyridine) was dissolved in ammonium bicarbonate (20 mM, pH 7.8) and acetonitrile (1:3(v/v)). This solution was then added to the peptide arrays. The CLIPS template bound to side-chains of two cysteines introduced in the solid-phase bound peptides on a 455-well plate (3 μl per well). The arrays were gently shaken in this solution for 30-60 minutes. Afterward, the peptide arrays were extensively washed with excess water and sonicated in disrupt-buffer (1% SDS/0.1% 2,2′-(Ethylenedioxy)diethanethiol in PBS, pH 7.2) at 70° C. for 30 minutes, followed by additional sonication in water for another 45 minutes. Peptides carrying T3 CLIPS were synthesized similarly, but with three cysteines.

The binding of antibodies to each of the synthesized peptides was tested using an ELISA based on the Biosynth platform. The peptide arrays were incubated with primary antibody solution under two conditions: either overnight at 4° C. with a small amount of solution on top of the array (NORMAL) or at room temperature in a larger volume with agitation (BOX). After washing, the peptide arrays were incubated for one hour at 25° C. with a 1/1000 dilution of an appropriate peroxidase-conjugated secondary antibody—either goat anti-human HRP conjugate (Southern Biotech, 2010-05) or rabbit anti-mouse IgG(H+L) HRP conjugate (Southern Biotech, 6175-05). Following another wash, the peroxidase substrate, ABTS (2,2′-azino-di-3-ethylbenzthiazoline sulfonate), and 20 μl/ml of 3% hydrogen peroxide (H2O2) were added. After an hour, the color development was measured and quantified using a charge-coupled device (CCD) camera and an image processing system. This approach allows for a comprehensive evaluation of antibody binding to synthetic peptide epitopes, enabling effective epitope mapping.

The epitopes identified for each anti-Siglec-9 antibody are shown in bold in Table 5.

TABLE 5
Anti-Siglec-9 Antibody Epitopes

| Clone | |
|---|---|
| CB6B3 | MLLLLLPLLWGRERAEGQTSKLLTMQSSVTVQEGLCVHVPCSF |
| | SYPSHGWIYPGPVVHGYWFREGANTDQDAPVATNNPARAVWEE |
| | TRDRFHLLGDPHTKNCTLSIRDARRSDAGRYFFRMEKGSIKWN |
| | YKHHRLSVNVTALTHRPNILIPGTLESGCPQNLTCSVPWACEQ |
| | GTPPMISWIGTSVSPLDPSTTRSSVLTLIPQPQDHGTSLTCQV |
| | TFPGASVTTNKTVHLNVSYPPQNLTMTVFQGDGTVSTVLGNGS |
| | SLSLPEGQSLRLVCAVDAVDSNPPARLSLSWRGLTLCPSQPSN |
| | PGVLELPWVHLRDAAEFTCRAQNPLGSQQVYLNVSLQSKATSG |
| | VTQG |
| ED2D5 | MLLLLLPLLWGRERAEGQTSKLLTMQSSVTVQEGLCVHVPCSF |
| | SYPSHGWIYPGPVVHGYWFREGANTDQDAPVATNNPARAVWEE |
| | TRDRFHLLGDPHTKNCTLSIRDARRSDAGRYFFRMEKGSIKWN |
| | YKHHRLSVNVTALTHRPNILIPGTLESGCPQNLTCSVPWACEQ |
| | GTPPMISWIGTSVSPLDPSTTRSSVLTLIPQPQDHGTSLTCQV |
| | TFPGASVTTNKTVHLNVSYPPQNLTMTVFQGDGTVSTVLGNGS |
| | SLSLPEGQSLRLVCAVDAVDSNPPARLSLSWRGLTLCPSQPSN |
| | PGVLELPWVHLRDAAEFTCRAQNPLGSQQVYLNVSLQSKATSG |
| | VTQG |
| mAbA | MLLLLLPLLWGRERAEGQTSKLLTMQSSVTVQEGLCVHVPCSF |
| | SYPSHGWIYPGPVVHGYWFREGANTDQDAPVATNNPARAVWEE |
| | TRDRFHLLGDPHTKNCTLSIRDARRSDAGRYFFRMEKGSIKWN |
| | YKHHRLSVNVTALTHRPNILIPGTLESGCPQNLTCSVPWACEQ |
| | GTPPMISWIGTSVSPLDPSTTRSSVLTLIPQPQDHGTSLTCQV |
| | TFPGASVTTNKTVHLNVSYPPQNLTMTVFQGDGTVSTVLGNGS |
| | SLSLPEGQSLRLVCAVDAVDSNPPARLSLSWRGLTLCPSQPSN |
| | PGVLELPWVHLRDAAEFTCRAQNPLGSQQVYLNVSLQSKATSG |
| | VTQG |

### Example 6: Binding Specificity of Anti-Siglec-9 Antibodies

Biolayer interferometry (BLI) and ELISA experiments were performed to assess the affinity and specificity of the generated anti-Siglec-9 antibodies.

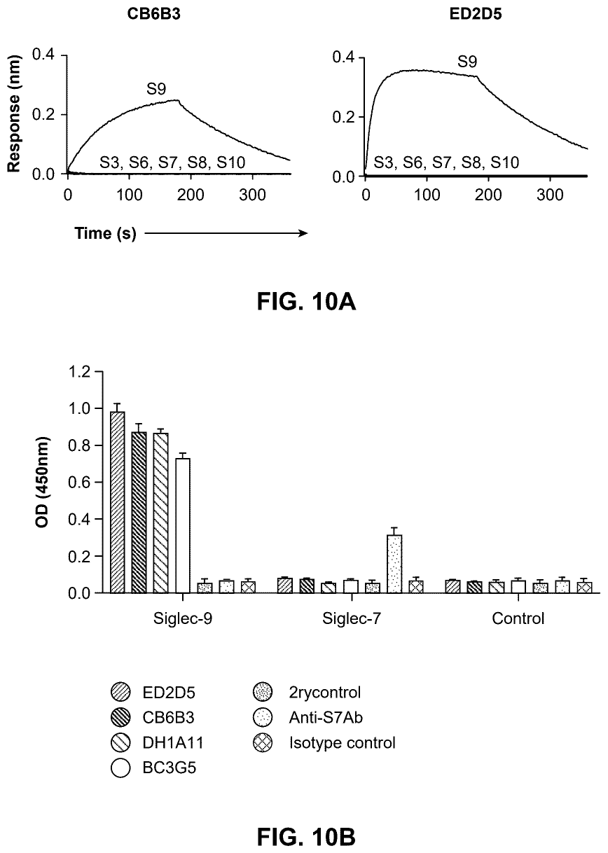

BLI experiments were performed to assess binding of the generated anti-Siglec-9 antibodies to Siglec family members Siglec-3, Siglec-6, Siglec-7, Siglec-8, Siglec-9, and Siglec-10. The antibodies were tested in Fab format at 250 nM. Antibody clones C1B613 and ED2D5 demonstrated binding to Siglec-9 but did not bind to Siglec-3, Siglec-6, Siglec-7, Siglec-8, 9, or Siglec-10 (FIG. 10A).

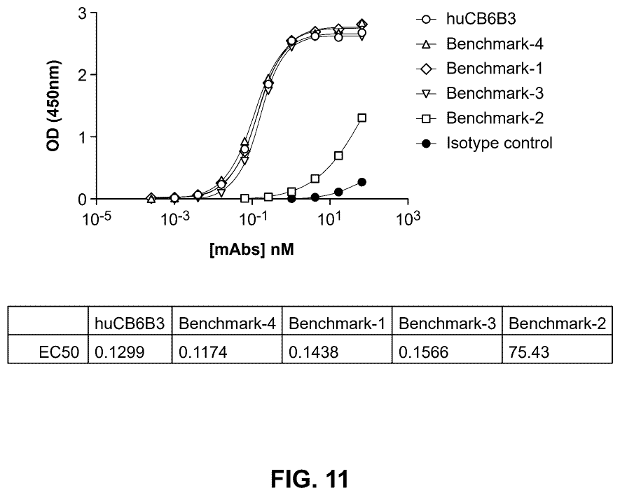

Next, the binding of anti-Siglec-9 monoclonal antibodies ED2D5, C1B613, BC3G5, and DH1A11 to the ectodomains of Siglec-9, Siglec-7, and a control was assessed using ELISA. An anti-Siglec-7 antibody control and isotype control were utilized. Antibodies ED2D5, C1B613, DH1A11, and 1BC3G5 demonstrated binding to Siglec-9 but not to Siglec-7 or a control, affirming their specificity for Siglec-9 (FIG. 10B). The anti-Siglec-7 antibody demonstrated binding only to Siglec-7, and the 2ry and isotype controls did not bind to any of Siglec-9, Siglec-7, or control. Optical density (GD) versus antibody concentration revealed a similar Siglec-9 binding profile for antibody clones huCB6B3, mAbA, 68D4, and Tf73, and little to no binding for clone 5C6 and an isotype control (FIG. 11). Antibody clones huCB6B3, mAbA, 68D4, and Tf73 also produced similar EC50 values.

To perform the ELISA experiments, 96-well plates were coated with recombinant Siglec-9 (SinoBiological, 30109-H03H) (100 μL) at 2 μg/mL in carbonate/bicarbonate buffer pH 9.6 overnight at 4° C., and washed with 250 μL of PBST (PBS, 0.1% Tween-20). Plates were blocked with blocking buffer (3% BSA in PBS) for 1 hour at room temperature, washed, and 100 μL of serially diluted anti-Siglec-9 antibodies-test antibodies, benchmarks, or control-starting at 66.64 nM (4-fold serial dilutions in PBST with 1% w/v non-fat milk), were added for 2 hours at RT. The plates were washed and incubated at room temperature with an anti-mouse IgG HRP detection antibody (1:5000, 1-hour, Anti-mouse IgG-HRP), followed by three washes of 250 μL of PBST, and two more with 400 μL of PBS, before the addition of 100 μL of TMB (Thermo Scientific) for 2-6 minutes. The reaction was stopped with 50 μL of Stop Solution (Thermo Scientific). The OD was measured at 450 nm in a multimode plate reader (Victor Nivo, PerkinElmer). OD values were plotted against antibody concentration, and data were analyzed using GraphPad Prism software.

### Example 7: Assessment of Siglec-9 Expression on Primary Macrophages

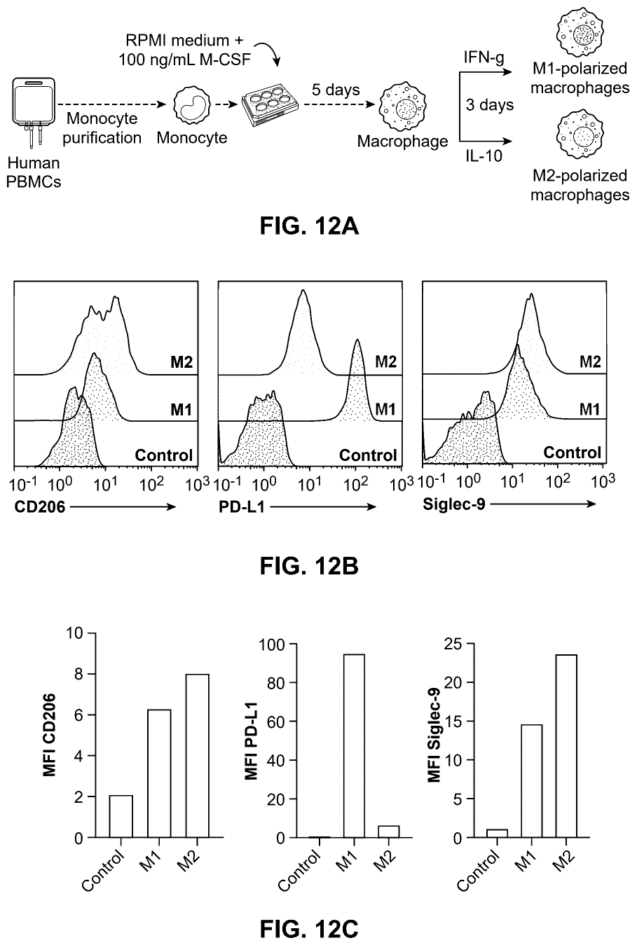

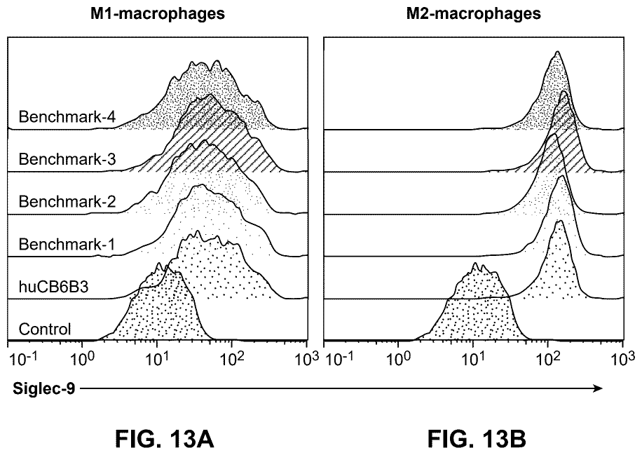

Flow cytometry analysis was utilized to assess the expression of Siglec-9, PD-L1, and CD206 on primary human macrophages. First, human CD14+ monocytes were differentiated and polarized into M1- or M2-like macrophages (FIG. 12A). Analyses of the M1- and M2-like macrophages demonstrated expression of CD206, with both populations having higher expression than a control (FIGS. 12B and 12C). M1-like macrophages strongly expressed PD-L1 at a much greater level than M2-like macrophages or a control. Both M1- and M2-like macrophages expressed Siglec-9, with M2-like macrophages having higher expression than M1-like macrophages. Antibody clones huCB6B3, mAbA, 5C6, 68D4, and Tf73 were then assessed for binding to M1- and M2-macrophages. All antibodies demonstrated binding to M1-like macrophages and bound to a greater extent than the control (FIG. 13A). All antibodies bound to M2-like macrophages to a greater extent than M1-like macrophages.

To perform these experiments, human peripheral blood mononuclear cells (PBMCs) were isolated from blood donors, who provided informed consent, using density gradient centrifugation. CD14⁺ monocytes were then purified from PBMCs utilizing a CD14 positive selection kit (Stemcell) according to the manufacturer's instructions. The isolated monocytes were differentiated into macrophages by culturing in RPMI 1640 medium supplemented with 10% FBS and 100 ng/mL macrophage colony-stimulating factor (M-CSF) for 5 days. After that, IFN-γ (100 ng/mL) or IL10 (100 ng/mL) were added to the culture medium to polarized macrophages to M1-like or M2-like, respectively.

Polarized M1- or M2-like macrophages were detached with EDTA 5 nM (100 ul) for 30 minutes, centrifuged at 450×G for 5 minutes and the supernatant was discarded. The antibody staining mix was prepared with anti-human Siglec-9 (clone huCB6B3 and benchmark antibodies), anti-human PD-L1 (329705-Biolegend), and anti-human CD206 (53-2069-42-eBioscience) and cells were resuspended in 100 μl of the mix. Cells were incubated for 40 minutes on ice, and 100 μl of staining buffer were added to each well, plates were centrifuged at 450×G for 5 minutes and supernatants were discarded. In some cases, secondary antibody was prepared: anti-human IgG-APC at 1:200 dilution, and was added (100 pl) to each well. Cells were incubated for 40 minutes on ice followed by addition of 100 μl of staining buffer to each well, plates were centrifuged at 450×G for 5 minutes to discard the supernatant. Cells were resuspended in 150 μl of staining buffer and acquired in the flow cytometer.

### Example 8: IFN-γ Production by Primary T Cells with Siglec-9 Blockade

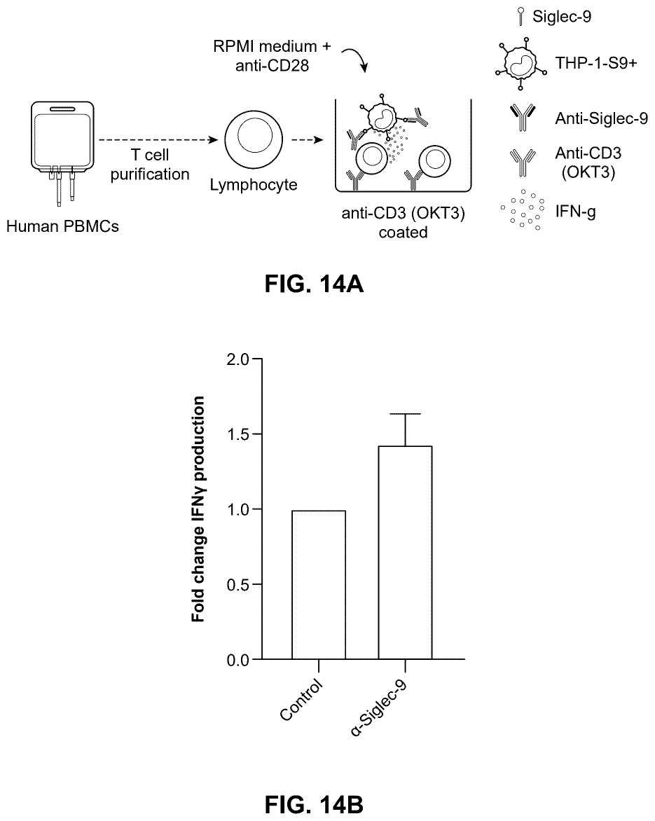

To assess the effect of Siglec-9 blockade on production of IFN-γ by T cells, primary human T cells were co-cultured with THP-1 Siglec-9⁺ cells in the presence of anti-Siglec-9 mAb clone huCB6B3 (FIG. 14A). ELISA quantification of IFN-γ in the supernatant after 72 hours revealed huCB6B3 increased IFN-γ production by primary human T cells relative to a control (FIG. 14B).

To perform these experiments, peripheral blood mononuclear cells (PBMCs) were isolated from healthy blood donors (with informed consent) using density gradient centrifugation. T cells were purified from PBMCs using a CD3-negative selection kit (Stemcell Technologies), following the manufacturer's protocol. Purified T cells were cultured in RPMI 1640 medium supplemented with 10% fetal bovine serum (FBS) in 96-well plates pre-coated with anti-CD3 monoclonal antibody (OKT3; Cat #16-0037-85, eBioscience).

THP-1 S9⁺ cells (20,000 per well) were pre-treated with 33.3 nM anti-Siglec-9 antibody (huCB6B3) for 30 minutes on ice. Treated THP-1 cells were added to T cells at a 1:7 ratio (THP-1:T cell). Anti-CD28 antibody (3.33 nM) was also added to each well to provide co-stimulation. After 72 hours of co-culture, supernatants were collected for IFN-γ quantification by ELISA. IFN-γ levels were measured using the Human IFN-γ DuoSet ELISA kit (Cat #DY285B, R&D Systems) according to the manufacturer's instructions. Briefly, a 96-well plate was coated overnight at room temperature (RT) with the capture antibody. After washing three times with Wash Buffer (0.05% Tween-20 in PBS), wells were blocked with 300 μL of Reagent Diluent (1% BSA in PBS) for 1 hour at RT. After another wash step, 100 μL of diluted samples were added and incubated for 2 hours at RT. Following three additional washes, 100 μL of detection antibody was added and incubated for 2 hours at RT. Wells were then washed again and incubated with 100 μL of Streptavidin-HRP for 20 minutes at RT. After washing, 100 μL of Substrate Solution was added and incubated for 20 minutes in the dark. The reaction was stopped with 50 μL of Stop Solution, and absorbance was measured at 450 nm using a microplate reader.

### Example 9: Evaluation of T Cell Cytotoxicity with Siglec-9 Blockade

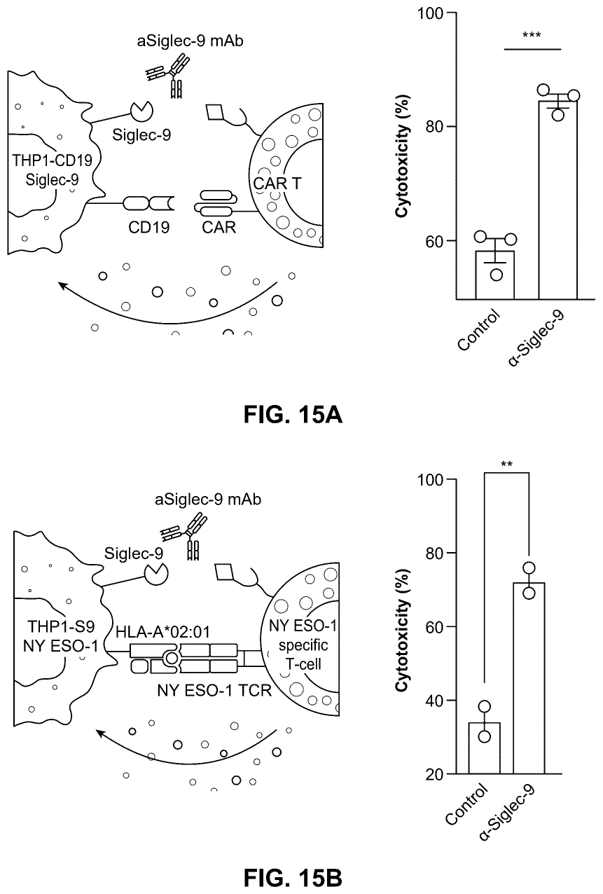

In order to determine the effect of Siglec-9 blockade on T cell cytotoxicity, THP-1 target cells expressing Siglec-9 and CD19 were co-cultured with anti-CD19 CAR-T cells in the presence of anti-Siglec-9 antibody clone huCB6B3 or a control. The blockade of Siglec-9 with huCB6B3 promoted CAR-T cytotoxicity against the target cells relative to the control (FIG. 15A). Similarly, antibody huCB6B3 promoted cytotoxicity of THP-1 target cells expressing NY-ESO by anti-NY-ESO-1 TCR-engineered human T cells relative to a control antibody (FIG. 15B).

To perform these experiments, THP-1 myeloid cells were engineered to overexpress either a truncated version of CD19 (CD19t) or HLA-A*02:01 presenting the NY-ESO-1 peptide. Both lines were also transduced to express full-length human Siglec-9 and tagged with GFP to enable flow cytometry-based quantification (TIP-1-CD19t-GFP-Siglec9 and THP-1-NY-ESO1-GFP-Siglec9). For the CD19 model, TIP-1-CD19t-GFP-Siglec9 cells were co-cultured with anti-CD19 CAR-T cells (19BBz) generated from three healthy donors at a 1:16 effector-to-target (E:T) ratio in 200 μl of non-supplemented AIM V medium (ThermoFisher, 12055091), with 5×10⁴ target cells per well.

In parallel, TIP-1-NY-ESO1-GFP-Siglec9 cells were co-cultured with anti-NY-ESO-1 TCR-engineered T cells from two healthy donors under the same conditions. Where indicated, the anti-Siglec-9 monoclonal antibody (clone huCB6B3) or isotype control was added at 10 μg/mL. After 16 hours, 20 μl of DAPI (1:2000 dilution) was added to each well to exclude dead cells, and viable GFP⁺ target cells were quantified on an Attune NxT flow cytometer (Thermo Fisher) equipped with an autosampler. Cytotoxicity was calculated as: (% cytotoxicity)=[(#alive GFP⁺ target cells in control wells−#alive GFP⁺ target cells in T cell co-culture)/#alive GFP⁺ target cells in control wells]×100.

### Example 10: Assessment of CAR-T Tumor Cell Cytotoxicity with Human Macrophages

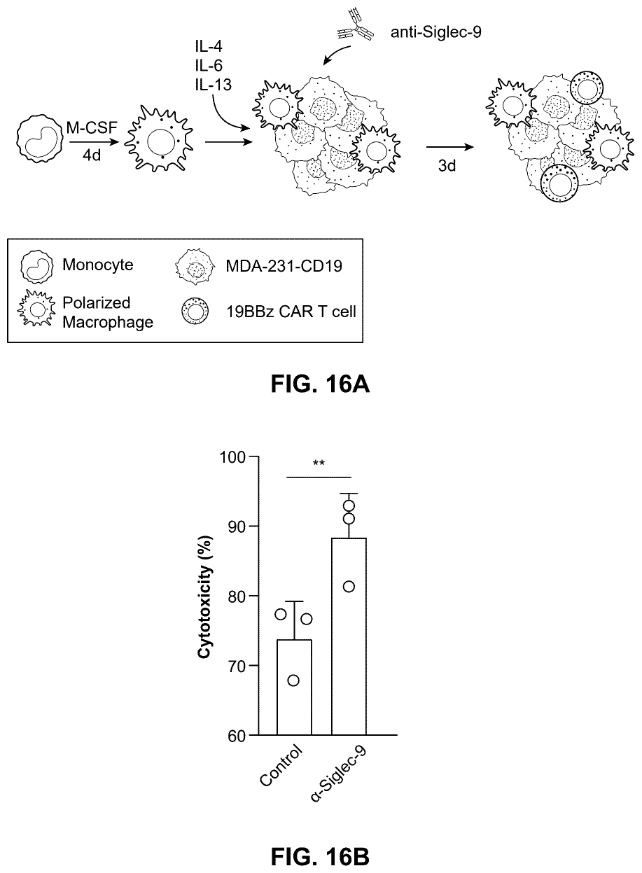

In order to determine the effect of Siglec-9 blockade on T cell cytotoxicity in the presence of human macrophages, CAR-T cells were co-cultured with human macrophages and tumor cells in the presence of anti-Siglec-9 clone huCB6B3 or a control human IgG (FIG. 16A). Siglec-9 blockade with huCB6B3 increased CAR-T cytotoxicity against tumor cells in the presence of human macrophages (FIG. 16B).

To model the immunosuppressive tumor microenvironment, a triple co-culture system was generated composed of anti-CD19 CAR-T cells (19BBz), MDA-MB-231 breast cancer cells engineered to express human CD19 and GFP-luciferase (MDA-MB-231-hCD19t-GFP-luc), and primary human macrophages. CD14⁺ monocytes were isolated from PBMCs and differentiated into M0 macrophages by culturing with M-CSF (50 ng/mL) for four days. These macrophages were then polarized by treatment with a cytokine cocktail (IL-4, IL-6, and IL-13, each at 20 ng/mL) to induce an immunosuppressive phenotype. Polarized macrophages were co-cultured with MDA-MB-231-hCD19t-GFP-luc tumor cells in black-walled, flat-bottom 96-well plates using DMEM/F12 Advanced medium (Gibco) supplemented with 1% FBS to reduce interference with glycan-Siglec interactions. Anti-Siglec-9 blocking antibody (clone huCB6B3) or control was added at 10 μg/mL. After a 72-hour incubation, 19BBz CAR-T cells were added at a ratio of 1:10:10 (CAR-T:tumor:macrophages), and co-cultures were maintained for an additional 72 hours. Cytotoxicity was measured by adding 50 μL of Bright-Glo luciferase substrate (Promega) and reading luminescence on a VICTOR Nivo plate reader (PerkinElmer). Percent cytotoxicity was calculated using the formula: [(1−RLU_sample/RLU_max)×100], where RLU_max represents luminescence from wells lacking CAR-T cells.

### Example 11: Characterization of the Expression of Siglec-9 in Human Cancers

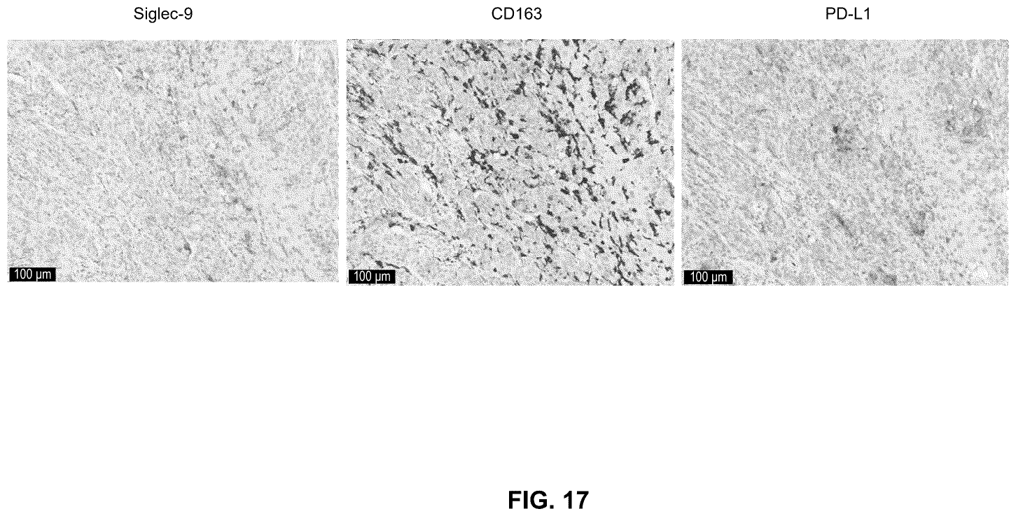

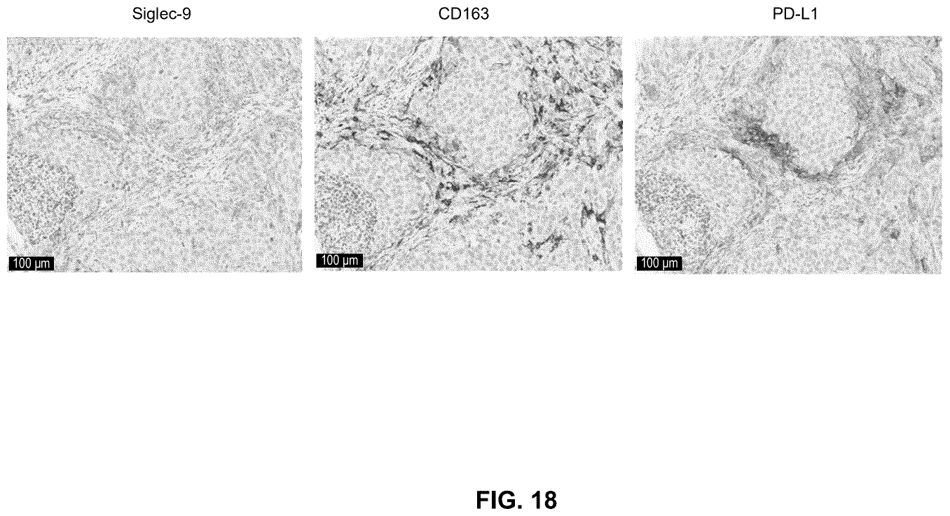

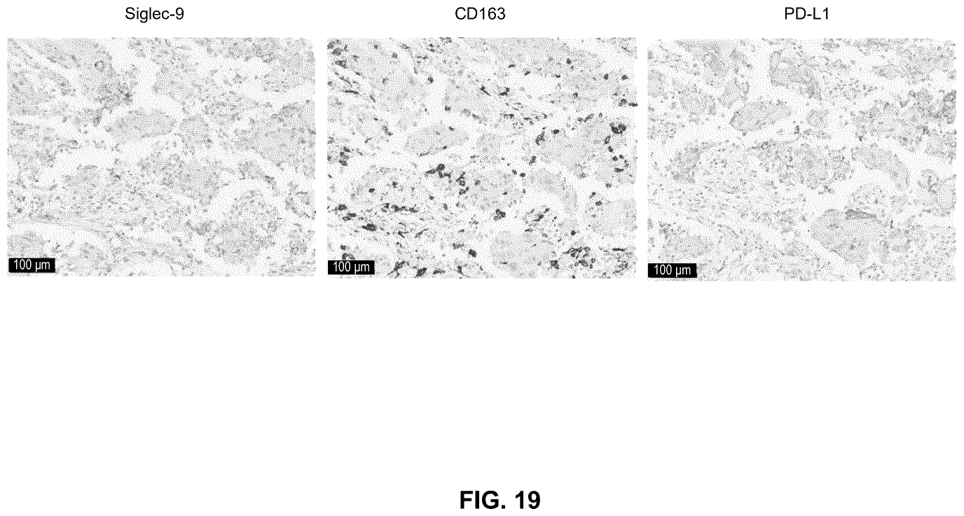

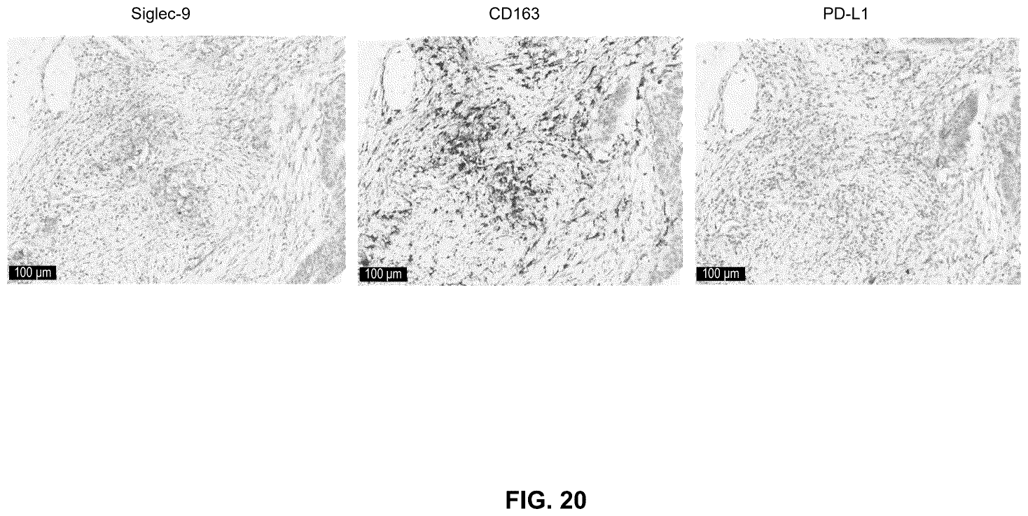

Immunohistochemistry was performed to characterize the expression of Siglec-9, CD163, and PD-L1 in human hepatocellular carcinoma (FIG. 17), breast cancer (FIG. 18), lung adenocarcinoma (FIG. 19), and rectum adenocarcinoma (FIG. 20).

Human Paraffin Embedded Tissue Arrays were acquired from https://www.tissuearray.com/tissue-arrays. The chromogen was DAB (Brown staining) and the counter staining for the nuclei was performed with hematoxylin (blue staining). The immunohistochemistry was performed on the automated platform Bond RX (Leica) using the detection kit Bond Refine polymer DAB. The tissue sections were prepared from the FFPE tissue blocks using a microtome set to cut at 4 μm. The tissue sections, approximately 4 μm thick, were mounted on superfrost plus glass slides and allowed to dry in the oven at least 1 h at 56-60° C. The slides were stored at room temperature until used. The primary antibody for Siglec-9 staining, polyclonal ref. 13377-1-AP—Proteintech, was first applied on the positive and negative cell pellets at a concentration of 1 μg/mL. The best staining was obtained with the epitope retrieval buffer of the Bond RX platform ER2 pretreatment buffer ER2 (20 minutes). Incubation: 45 min in Leica antibody diluent at a concentration of 1 μg/mL. The immunostained slides were digitized using a Hamamatsu XR slide scanner slide scanner at 20× magnification.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1. An antibody or antigen-binding fragment thereof that binds to Siglec-9 protein, wherein the antibody or antigen-binding fragment thereof comprises:
(i) a heavy chain variable complementarity determining region 1 (CDR-H1) comprising the amino acid sequence of SEQ ID NO: 5, a CDR-H2 comprising the amino acid sequence of SEQ ID NO: 6, a CDR-H3 comprising the amino acid sequence of SEQ ID NO: 7, a light chain variable complementarity determining region 1 (CDR-L1) comprising the amino acid sequence of SEQ ID NO: 8, a CDR-L2 comprising the amino acid sequence of SEQ ID NO: 9, and a CDR-L3 comprising the amino acid sequence of SEQ ID NO: 10; or
(ii) a heavy chain variable complementarity determining region 1 (CDR-H1) comprising the amino acid sequence of SEQ ID NO: 13, a CDR-H2 comprising the amino acid sequence of SEQ ID NO: 14, a CDR-H3 comprising the amino acid sequence of SEQ ID NO: 15, a light chain variable complementarity determining region 1 (CDR-L1) comprising the amino acid sequence of SEQ ID NO: 16, a CDR-L2 comprising the amino acid sequence of SEQ ID NO: 17, and a CDR-L3 comprising the amino acid sequence of SEQ ID NO:18.

2. (canceled)

3. (canceled)

4. The antibody or antigen-binding fragment thereof of claim 1, wherein the antibody or antigen-binding fragment thereof comprises:
(i) a heavy chain variable region and a light chain variable region comprising the amino acid sequences of SEQ ID NOs: 3 and 4, respectively;
(ii) a heavy chain variable region and a light chain variable region comprising the amino acid sequences of SEQ ID NOs: 11 and 12, respectively; or
(iii) a heavy chain variable region and a light chain variable region comprising the amino acid sequences of SEQ ID NOs: 19 and 20, respectively.

5.-8. (canceled)

9. The antibody or antigen-binding fragment thereof of claim 1, wherein the antibody or antigen-binding fragment thereof is a murine antibody, a humanized antibody, a bispecific antibody, a monoclonal antibody, a multivalent antibody, a conjugated antibody, a human antibody, or a chimeric antibody or antigen-binding fragment thereof.

10.-12. (canceled)

13. A polynucleotide comprising a nucleic acid molecule encoding the VH and the VL of the antibody or antigen-binding fragment thereof of claim 1.

14. An isolated vector comprising one or more of the polynucleotides of claim 13.

15. A host cell comprising the vector of claim 14.

16.-21. (canceled)

22. A pharmaceutical composition comprising the antibody or antigen-binding fragment thereof of claim 1 and a pharmaceutically acceptable carrier.

23.-25. (canceled)

26. A method of treating or preventing cancer in a subject in need thereof comprising administering to the subject a therapeutically effect amount of the antibody or antigen-binding fragment of claim 1.

27. (canceled)

28. (canceled)

29. The method of claim 26, wherein the antibody or antigen-binding fragment is administered in combination with an additional therapeutic agent for treating, preventing and/or diagnosing the cancer, and wherein the therapeutic agent is a chemotherapeutic agent or an immunotherapy.

30.-36. (canceled)

37. A bispecific antibody comprising:
a) a first antigen-binding portion that binds to Siglec-9 comprising:
a heavy chain variable complementarity determining region 1 (CDR-H1) comprising the amino acid sequence of SEQ ID NO: 5, a CDR-H2 comprising the amino acid sequence of SEQ ID NO: 6, a CDR-H3 comprising the amino acid sequence of SEQ ID NO: 7, a light chain variable complementarity determining region 1 (CDR-L1) comprising the amino acid sequence of SEQ ID NO: 8, a CDR-L2 comprising the amino acid sequence of SEQ ID NO: 9, and a CDR-L3 comprising the amino acid sequence of SEQ ID NO: 10; or
a heavy chain variable complementarity determining region 1 (CDR-H1) comprising the amino acid sequence of SEQ ID NO: 13, a CDR-H2 comprising the amino acid sequence of SEQ ID NO: 14, a CDR-H3 comprising the amino acid sequence of SEQ ID NO: 15, a light chain variable complementarity determining region 1 (CDR-L1) comprising the amino acid sequence of SEQ ID NO: 16, a CDR-L2 comprising the amino acid sequence of SEQ ID NO: 17, and a CDR-L3 comprising the amino acid sequence of SEQ ID NO:18; and
b) a second antigen-binding portion that binds to a second antigen.

38. (canceled)

39. (canceled)

40. The bispecific antibody of claim 37, wherein the first antigen-binding portion comprises:
(i) a heavy chain variable region and a light chain variable region comprising the amino acid sequences of SEQ ID NOs: 3 and 4, respectively;
(ii) a heavy chain variable region and a light chain variable region comprising the amino acid sequences of SEQ ID NOs: 11 and 12, respectively; or
(iii) a heavy chain variable region and a light chain variable region comprising the amino acid sequences of SEQ ID NOs: 19 and 20, respectively.

41. The bispecific antibody of claim 37, wherein the second antigen is a cell surface protein that is enriched on blood-brain barrier endothelial cells selected from the group consisting of transferrin receptor, insulin receptor, insulin-like growth factor receptor, low-density lipoprotein receptor related proteins 1 and 2, diphtheria toxin receptor, CRM197, a llama single domain antibody, TMEM 30(A), a protein transduction domain, TAT, Syn-B, penetratin, a poly-arginine peptide, Angiopep peptides, other cell surface proteins that are enriched on blood-brain barrier endothelial cells.

42. The bispecific antibody of claim 37, wherein the second antigen is a protein expressed on immune cells selected from the group consisting of PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIMI, TIM3, TIM4, A2AR, LAG3, DR-5, CD2, CD5, TREM1, TREM2, CD39, CD73, CSF-1 receptor, Siglec-7, Siglec-10, Siglec-15, SIRPα, CD47, LILRB1, LILRB2, NKG2A, TGFBR, IDO1, STING, and phosphatidylserine.

43. (canceled)

44. The bispecific antibody of claim 37, wherein each of the first and second antigen-binding portions thereof is independently from a murine antibody, a humanized antibody, a monoclonal antibody, a conjugated antibody, a human antibody, or a chimeric antibody or antigen-binding fragment thereof.

45.-62. (canceled)

63. A pharmaceutical composition comprising the bispecific antibody of claim 37 and a pharmaceutically acceptable carrier.

64.-74. (canceled)

75. A conjugate comprising an antibody or antigen-binding fragment thereof that binds to Siglec-9 protein coupled to an active agent.

76. The conjugate of claim 75, wherein the antibody or antigen-binding fragment thereof comprises an antibody or antigen-binding fragment, wherein the antibody or antigen-binding fragment thereof comprises:
(i) a heavy chain variable complementarity determining region 1 (CDR-H1) comprising the amino acid sequence of SEQ ID NO: 5, a CDR-H2 comprising the amino acid sequence of SEQ ID NO: 6, a CDR-H3 comprising the amino acid sequence of SEQ ID NO: 7, a light chain variable complementarity determining region 1 (CDR-L1) comprising the amino acid sequence of SEQ ID NO: 8, a CDR-L2 comprising the amino acid sequence of SEQ ID NO: 9, and a CDR-L3 comprising the amino acid sequence of SEQ ID NO: 10: or
(ii) a heavy chain variable complementarity determining region 1 (CDR-H1) comprising the amino acid sequence of SEQ ID NO: 13, a CDR-H2 comprising the amino acid sequence of SEQ ID NO: 14, a CDR-H3 comprising the amino acid sequence of SEQ ID NO: 15, a light chain variable complementarity determining region 1 (CDR-L1) comprising the amino acid sequence of SEQ ID NO: 16, a CDR-L2 comprising the amino acid sequence of SEQ ID NO: 17, and a CDR-L3 comprising the amino acid sequence of SEQ ID NO:18.

77. (canceled)

78. (canceled)

79. The conjugate of claim 75, wherein the active agent is an immunomodulatory agent, a cytotoxic agent, a radiolabeled agent, or any combination thereof.

80. (canceled)

81. A pharmaceutical composition comprising the conjugate of claim 75 and a pharmaceutically acceptable carrier.

82.-93. (canceled)

94. The CAR of claim 96, wherein the intracellular domain comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

95. (canceled)

96. A cell expressing an isolated chimeric antigen receptor (CAR), the CAR comprising:
a) an extracellular portion comprising an antibody or antigen-binding fragment that binds to Siglec-9 protein comprising the antibody or antigen-binding fragment of claim 1;
b) a transmembrane domain; and
c) an intracellular signaling domain.

97.-109. (canceled)


SEQUENCE LISTING (21 sequences)

SEQ ID NO: 1 - protein, 463 aa, Homo sapiens
  source 1..463: note = Human Siglec-9
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 2 - protein, 327 aa, synthetic construct
  source 1..327: note = Recombinant Siglec-9
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSG

SEQ ID NO: 3 - protein, 120 aa, synthetic construct
  source 1..120: note = ED2D5 heavy chain
EVQLVESGGGLVKPGGSLKLSCAASGFTFSDYYMYWVRQTPEKRLEWVATISDGGSDTYYPDNMKGRFTISRDNAKNNLYLQMSSLKSEDTAMYYCTRDPDYDDGRFAYWGQGTLVTVSA

SEQ ID NO: 4 - protein, 107 aa, synthetic construct
  source 1..107: note = ED2D5 light chain
DIVMTQSHKFMSTSVGDRVSITCKASQDVGTVVAWYQQKSGQSPKLLIYWSSTRHTGVPDRFTGSGSGTDFTLTISNVQSEDLADYFCQQYIDYPLSFGAGTKLELK

SEQ ID NO: 5 - protein, 5 aa, synthetic construct
  source 1..5: note = ED2D5 CDR-H1
DYYMY

SEQ ID NO: 6 - protein, 17 aa, synthetic construct
  source 1..17: note = ED2D5 CDR-H2
TISDGGSDTYYPDNMKG

SEQ ID NO: 7 - protein, 11 aa, synthetic construct
  source 1..11: note = ED2D5 CDR-H3
DPDYDDGRFAY

SEQ ID NO: 8 - protein, 11 aa, synthetic construct
  source 1..11: note = ED2D5 CDR-L1
KASQDVGTVVA

SEQ ID NO: 9 - protein, 7 aa, synthetic construct
  source 1..7: note = ED2D5 CDR L2
WSSTRHT

SEQ ID NO: 10 - protein, 9 aa, synthetic construct
  source 1..9: note = ED2D5 CDR L3
QQYIDYPLS

SEQ ID NO: 11 - protein, 118 aa, synthetic construct
  source 1..118: note = CB6B3 heavy chain
EVQLQQSGAEVVRPGALVKLSCKAAGFNIKDYFIHWVKQRPEQGLEWIGLIDPENDNTMYDPRFQVKASLTADTSSNTAYLQLSRLTSEDAAVYYCASTVVARSFDYWGQGTTLTVSS

SEQ ID NO: 12 - protein, 107 aa, synthetic construct
  source 1..107: note = CB6B3 light chain
SIVMTQTPKFLLVSAGDRVTITCKASQSVSNDVAWYQQKSGQSPKLLIYYTSNRYTGVPDRFTGSGYGTDFTFTISTVQAEDLAVYFCQQDFRSPFTFGAGTKLELK

SEQ ID NO: 13 - protein, 5 aa, synthetic construct
  source 1..5: note = CB6B3 CDR-H1
DYFIH

SEQ ID NO: 14 - protein, 17 aa, synthetic construct
  source 1..17: note = CB6B3 CDR-H2
LIDPENDNTMYDPRFQV

SEQ ID NO: 15 - protein, 9 aa, synthetic construct
  source 1..9: note = CB6B3 CDR-H3
TVVARSFDY

SEQ ID NO: 16 - protein, 11 aa, synthetic construct
  source 1..11: note = CB6B3 CDR-L1
KASQSVSNDVA

SEQ ID NO: 17 - protein, 7 aa, synthetic construct
  source 1..7: note = CB6B3 CDR L2
YTSNRYT

SEQ ID NO: 18 - protein, 9 aa, synthetic construct
  source 1..9: note = CB6B3 CDR L3
QQDFRSPFT

SEQ ID NO: 19 - protein, 118 aa, Homo sapiens
  source 1..118: note = Human IgG CB6B3 heavy chain
EVQLVQSGAEVKKPGATVKISCKVSGYTFTDYFIHWVQQAPGKGLEWMGLIDPENDNTMYDPRFQVRVTITADTSTDTAYMELSSLRSEDTAVYYCASTVVARSFDYWGQGTTVTVSS

SEQ ID NO: 20 - protein, 107 aa, Homo sapiens
  source 1..107: note = Human IgG CB6B3 light chain
AIQMTQSPSSLSASVGDRVTITCKASQSVSNDVAWYQQKPGKAPKLLIYYTSNRYTGVPSRFSGSGSGTDFTLTISSLQPEDFATYYCQQDFRSPFTFGGGTKVEIK

SEQ ID NO: 21 - protein, 330 aa, Homo sapiens [sequence omitted]